(19) United States
(12) Patent Application Publication
(10) Pub. No.: US 2018/0303970 A1
(43) Pub. Date: Oct. 25, 2018

(54) COMPOSITIONS AND METHODS FOR HEALING WOUNDS

(71) Applicant: VeriGraft AB, Goteborg (SE)

(72) Inventor: Suchitra Sumitran-Holgersson, Goteborg (SE)

(21) Appl. No.: 15/769,128

(22) PCT Filed: Oct. 28, 2016
(86) PCT No.: PCT/EP2016/076178

§ 371 Date: Apr. 18, 2018

Related U.S. Application Data
(60) Provisional application No. 62/249,587, filed on Nov. 2, 2015.

(51) Int. Cl. A61L 26/00; A61P 17/02

(52) U.S. Cl. CPC A61L 26/0057; A61L 26/0023; A61L 26/0066; A61L 26/0052; A61L 2430/34; A61L 26/0047; A61L 26/008; A61P 17/02; A61L 2300/414; A61L 26/0033

(57) ABSTRACT

The present disclosure relates to a composition of an engineered biomaterial including extracellular matrix components of a mammalian tissue and a polymer; method of wound healing; and methods delivering therapeutic agents, growth factors, or hydration for wound healing to a subject in need thereof.

### RELATED APPLICATIONS

This application claims priority to U.S. Provisional Patent Application No. 62/249,587, filed Nov. 2, 2015, the disclosure of which is incorporated by reference in its entirety for all purposes.

### BACKGROUND OF THE DISCLOSURE

Wound healing, as a normal biological process in the human body, is achieved through four precisely and highly programmed phases: hemostasis, inflammation, proliferation, and remodeling. For a wound to heal successfully, all four phases must occur in the proper sequence and time frame. Many factors can interfere with one or more phases of this process, thus causing improper or impaired wound healing.

Wounds that exhibit impaired healing, including delayed acute wounds and chronic wounds, generally have failed to progress through the normal stages of healing. Such wounds frequently enter a state of pathologic inflammation due to a postponed, incomplete, or uncoordinated healing process. Most chronic wounds are ulcers that are associated with ischemia, diabetes mellitus, venous stasis disease, or pressure. Non-healing wounds affect about 3 to 6 million people in the United States, with persons 65 years and older accounting for 85% of these events. Non-healing wounds result in enormous health care expenditures, with the total cost estimated at more than $3 billion per year.

In adult humans, optimal wound healing involves the following the events: (1) rapid hemostasis; (2) appropriate inflammation; (3) mesenchymal cell differentiation, proliferation, and migration to the wound site; (4) suitable angiogenesis; (5) prompt re-epithelialization (re-growth of epithelial tissue over the wound surface); and (6) proper synthesis, cross-linking, and alignment of collagen to provide strength to the healing tissue.

Various cell-based therapies and tissue engineering strategies have been developed to treat patients suffering from chronic wounds or acute skin trauma. None are, however, optimal in achieving durable wound healing of chronic or acute wounds. To achieve durable wound closure, the formation of a functional vascular network is important for the regeneration of wound beds. The current disclosure provides an improved composition for use in healing wound.

### BRIEF SUMMARY OF THE DISCLOSURE

The current disclosure provides, inter alia, a composition (e.g., an engineered biomaterial) including ECM components of a mammalian tissue and a polymer for application to a wound; a method of healing a wound of a subject in need thereof, the method including applying the composition (e.g., an engineered biomaterial); a method of delivering a therapeutic agent to a wound of a subject in need thereof, including applying the composition (e.g., an engineered biomaterial); a method of delivering a growth factor to a wound of a subject in need thereof, including applying a composition (e.g., an engineered biomaterial) including extracellular matrix (ECM) components of a mammalian tissue, a polymer, and a growth factor to a wound of the subject; and a method of hydrating a wound of a subject in need thereof, the method including applying a composition (e.g., an engineered biomaterial) including extracellular matrix (ECM) components of a mammalian tissue and a polymer to a wound of the subject. The mammalian tissue may be a decellularized mammalian tissue. The components of the ECM are or may be isolated and/or purified from a tissue of a mammal (e.g., human) or generated using recombinant DNA technology involving gene or gene fragments of a mammal (e.g., human) encoding the respective ECM component (e.g., collagens, elastins, laminins, glycosaminoglycans, proteoglycans, antimicrobials, chemoattractants, cytokines, and growth factors) and expressed in from a suitable expression system (prokaryotic or eukaryotic (e.g., insect or mammalian cells)), and subsequently isolated and/or purified by suitable method in the art.

The ECM components in the composition (e.g., an engineered biomaterial) may be from a mammalian tissue, e.g., epithelial and/or connective tissue, and may be obtained from the skin, placenta, and/or umbilical cord. The composition (e.g., an engineered biomaterial) may be a gel composition. The polymer of the composition (e.g., an engineered biomaterial) may include a disaccharide polymer, e.g., hyaluronic acid. The composition (e.g., an engineered biomaterial) may further include blood cells or platelet rich plasma. The composition (e.g., an engineered biomaterial) may include human peripheral blood mononuclear cells. The ECM components may include collagen, elastin, and/or sulfated glycosaminoglycans (GAGs). The ECM components may be admixed with a synthetic polymer.

The mammalian tissue of the composition (e.g., an engineered biomaterial) may be obtained from a mammal, e.g., pig, cow, lamb, goat, sheep, or primate (e.g., human). The mammalian tissue may be a decellularized mammalian tissue. The mammal may be a pig. The mammal, e.g, the pig, may be a knockout mutant for the α-Gal (Galα1,3-Galβ1-4-GlcNAc-R) epitope.

The method of healing a wound includes healing an acute or chronic wound, or a skin wound, including a skin tear, friction, a closed impact surgical wound, a skin abrasion, a burn, a skin incision, a skin laceration, a skin contusion, a skin puncture, a pressure ulcer, a venous ulcer, an arterial ulcer, a neuropathic/diabetic wound, lymphedema, or a surgical site incision.

Other features and advantages of the disclosure will be apparent from the following detailed description and claims.

Unless noted to the contrary, all publications, references, patents and/or patent applications reference herein are hereby incorporated by reference in their entirety for all purposes.

### BRIEF DESCRIPTION OF THE DRAWINGS

FIGS. 1A-1G are gross morphology and histology images of pig skin. Gal/gal knockout native pig skin with epidermal side up (FIG. 1A) and dermal side (FIG. 1B) up demonstrated a pinkish color, presence of hair, and subcutaneous fat. FIG. 1C shows hematoxylin and eosin staining of native pig skin showing the presence of nuclei (blue) and cellular material. FIGS. 1D and 1E show images of decellularized pig skin after 9 days of decellularization, and the skin was pale and white. FIG. 1F shows hematoxylin and eosin images staining of decellularized pig skin with a complete absence of nuclei and cellular material. FIG. 1G shows an image of the gel prepared using decellularized pig skin containing hyaluronic acid and keratinocyte medium (scale bar—50 μm).

FIGS. 2A-2F shows a bar graph and images of the characterization of decellularized pig skin. FIG. 2A is a bar graph showing the presence of residual extracellular matrix components in decellularized pig skin powder despite removal of cells. FIG. 2B is an image showing masson's trichrome (MT) staining of native (upper panel) and decellularized (lower panel) pig skin collagen (blue). FIG. 2C is an image showing Verhoeff-Van Gieson (VVG) staining for elastin (black) in (upper panel) native and (lower panel) decellularized pig skin. FIG. 2D is an optical microscopy picture of powdered pig skin showing a filamentous, white macroscopic appearance which was transparent to light. FIGS. 2E and 2F are scanning electron micrographs showing that the pig skin powder consisted of ribbon-like fibres that varied in shape and size. The fibres were irregularly wrinkled and twisted, forming easily dispersible bundles (scale bar—200 μm).

FIGS. 3A-3D are a series of images showing the gross morphology of wound healing of a full-thickness cutaneous wound in nude mice. Healing was found to be significantly accelerated in mice treated with pig skin gel (PSG) only (FIG. 3C) or PSG+human peripheral blood mononuclear cells (PSG+hPBMC) (FIG. 3D) at day 15 as compared to untreated (FIG. 3A), or those treated with hyaluronic acid (HA) (FIG. 3B). In addition, a dark pink colored scar was still observed in the healed skin of the untreated group due to excessive contraction at day 25 and an elongated scar was also observed in the animals treated with hyaluronic acid (FIG. 3B) and keratinocyte medium, while a remarkably clear and almost completely healed skin with a slight or no scar was seen in animals treated with PSG (FIG. 3C) or PSG+hPBMC (FIG. 3D).

FIGS. 4A-4D is a series of images of hematoxylin and eosin staining of skin biopsies showing wound healing progression in nude mice. The progression in healing of a full-thickness cutaneous untreated wound; or treated with hyaluronic acid (HA) (FIG. 4B); or pig skin gel (PSG only) (FIG. 4C) or PSG+human peripheral blood mononuclear cells (PSG+hPBMC) (FIG. 4D) was studied by histology. As seen on day 5 after the operation, animals in all groups exhibited abundant inflammatory cells. The wounds in the control groups (untreated (FIG. 4A) and HA (FIG. 4B)) were distinguishable from adjacent tissues even at day 25, and no clear dermis layer was observed, while in the PSG only (FIG. 4C) and PSG+hPBMC (FIG. 4D) groups, keratin+dermal layers were clearly observed. While the restored skin of these animals showed a similar structure to that of normal skin, the dermal layer in the control group was still incomplete. The arrows indicate the wound area in all pictures and host skin is seen to the right of this region (scale bar—200 μm).

FIGS. 5A-5E are images and a bar graph showing collagen staining of wounds in untreated and pig skin gel treated nude mice. MT staining of the wounds in untreated (FIG. 5A) and HA treated (FIG. 5B) animals showed weaker staining of collagen (blue), while animals treated with PSG only (FIG. 5C) and PSG+hPBMC (FIG. 5D) showed strong staining for collagen at day 25 of wound healing. Collagen measurement in the regenerated skin on day 25, showed significantly increased expression in the animals treated with PSG+hPBMC as compared to untreated mice, p<0.05; (scale bar—100 μm) (FIG. 5E).

FIGS. 6A-6F are images showing detection of human cells in the wounds of animals treated with pig skin gel and human peripheral blood mononuclear cells. Presence of human cells in the epidermis and dermis of animals treated with pig skin gel (PSG) and human peripheral blood mononuclear cells was detected using a human specific mitochondrial antibody. No positive cells were found in the untreated animals (FIG. 6A) and no background staining was observed in the negative control (FIG. 6B). The specificity of the antibody was demonstrated in a positive control using a human liver tissue (FIG. 6C). Presence of positively stained cells in the epidermis and dermis of skin biopsies (black arrows) taken from animals treated with PSG+hPBMC demonstrated the presence of human cells on day 5 of wound healing (FIGS. 6D and 6E). Skin biopsies taken from the same animals showed the presence of human cells in newly formed blood vessels during wound healing; (scale bar—25 μm) (FIG. 6F).

FIGS. 7A-7G are images showing the detection of human blood vessels in wounds of animals treated with pig skin gel and human blood cells. Distribution and density of newly formed blood vessels were assessed using immunofluorescence. A mouse specific CD31 antibody (green) for mouse endothelial cells was used to detect host blood vessels on days 5 and 10 (FIGS. 7A and 7D). To determine whether human cells also contributed to neovascularization on days 5 and 10, a human specific CD31 antibody (red) was used (FIGS. 7B and 7E). FIGS. 7C and 7F are the merged pictures of FIGS. 7A and 7B, and 7D and 7E, respectively. As compared to control animals (FIG. 7G), markedly increased numbers of host and human blood vessels staining for mouse CD31 (green) and human CD31 (red) respectively were observed on day 5 in animals treated with PSG and human cells (upper panel). On day 10, however a lower number of human blood vessels were observed (lower panel).

### DETAILED DESCRIPTION OF THE DISCLOSURE

Provided herein, inter alia, is a composition (e.g., an engineered biomaterial) including ECM components of a mammalian tissue and a polymer; a method of healing a wound of a subject in need thereof, the method including applying the composition (e.g., an engineered biomaterial); and a method of delivering a therapeutic agent to a wound of a subject in need thereof, including applying the composition (e.g., an engineered biomaterial).

The mammalian tissue may be a decellularized mammalian tissue.

### Definitions

The following definitions are included for the purpose of understanding the present subject matter and for constructing the appended patent claims. Abbreviations used herein have their conventional meaning within the chemical and biological arts.

The term, “biomaterial” or “engineered biomaterial” as used in this disclosure may refer to any matter, surface, or construct that interacts with living systems, that may be naturally occurring or synthetically made. Thus, the term “biomaterial”, as used herein, refers to a substance that has been engineered to take a form which, alone or as part of a complex system, is used to direct, by control of interactions with components of living systems, the course of any therapeutic or diagnostic procedure. The term “one or more of” as used in this disclosure refers to a single component or alternatively a combination of two or more components.

The term, “gel” as used in this disclosure may refer to a material which is not a readily flowable liquid and not a solid, i.e., semi-solid. Gels may be formed from naturally occurring or synthetic materials. In embodiments, the gel may be a formed starting from a skin decellularized to prepare a powder, which includes extracellular matrix components.

The term “Extracellular Matrix” or “ECM” may refer to a natural or artificial scaffolding for cell growth. Natural ECMs (ECMs found in multicellular organisms, such as mammals and humans) are complex mixtures of structural and non-structural biomolecules, including, but not limited to, collagens, elastins, laminins, glycosaminoglycans, proteoglycans, antimicrobials, chemoattractants, cytokines, and growth factors. In mammals, ECM often comprises about 90% collagen, in its various forms. The composition and structure of ECMs vary depending on the source of the tissue. For example, small intestine submucosa (SIS), urinary bladder matrix (UBM) and liver stroma ECM each differ in their overall structure and composition due to the unique cellular niche needed for each tissue, and further characteristics and details as described in U.S. Pat. No. 8,361,503, incorporated herein by reference. Components of the ECM may be isolated and/or purified from a tissue, e.g., a decellularized tissue, of a mammal (e.g., human) or generated using recombinant DNA technology involving gene or gene fragments of a mammal (e.g., human) encoding the respective ECM component (e.g., collagens, elastins, laminins, glycosaminoglycans, proteoglycans, antimicrobials, chemoattractants, cytokines, and growth factors) and expressed in from a suitable expression system (prokaryotic or eukaryotic (e.g., insect or mammalian cells)), and subsequently isolated and/or purified by suitable method in the art.

The term “decellularized” is used in this disclosure to mean that physical, chemical, or enzymatic means, or any combination thereof, has removed the cellular component of vascular tissue thereof. The remaining decellularized vascular tissue comprises the extracellular matrix of the native vascular tissue and may include, but is not limited to, elastin, collagen, fibrin, and other extracellular proteins or non-proteinaceous compounds found in vascular tissue; or any combination thereof known to one of ordinary skill in the art, as described in U.S. Pat. No. 7,060,022, incorporated herein by reference.

The term “hyaluronic acid” or “HA” as used in this disclosure refers to a member of a class of polymers known as glycosaminoglycans. HA is a long chain linear polysaccharide and is usually present as the sodium salt which has a molecular formula of (C₁₄H₂₀NNaO₁₁)ₙ where n can vary according to the source, isolation procedure, and method of determination. However, molecular weights of up to 14×10⁶ have been reported, and previously described in U.S. Pat. No. 6,703,444.

The term “polymer” as referred to in this disclosure is meant as a molecule, or macromolecule, composed of many repeated subunits. Because of their broad range of properties, both synthetic and natural polymers play an essential and ubiquitous role in everyday life. Polymers range from familiar synthetic plastics such as polystyrene to natural biopolymers such as DNA and proteins that are fundamental to biological structure and function. Polymers, both natural and synthetic, are created via polymerization of many small molecules, known as monomers. Their consequently large molecular mass relative to small molecule compounds produces unique physical properties, including toughness, viscoelasticity, and a tendency to form glasses and semicrystalline structures rather than crystals. Exemplary natural polymeric materials include shellac, amber, wool, silk, rubber, and cellulose. Non-natural (e.g., synthetic) polymers include synthetic rubber, phenol formaldehyde resin, neoprene, nylon, polyvinyl chloride, polystyrene, polyethylene, polypropylene, and silicone.

Additional synthetic polymers that can be used include biodegradable polymers such as poly(lactide) (PLA), poly(glycolic acid) (PGA), poly(lactide-co-glycolide) (PLGA), poly(caprolactone), polycarbonates, polyamides, polyanhydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, Teflon®, and nylon. A non-absorbable polyvinyl alcohol sponge is available commercially as Ivalon™, from Unipoint Industries. Methods for making this material are described in U.S. Pat. No. 2,609,347 to Wilson; U.S. Pat. No. 2,653,917 to Hammon, U.S. Pat. No. 2,659,935 to Hammon, U.S. Pat. No. 2,664,366 to Wilson, U.S. Pat. No. 2,664,367 to Wilson, and U.S. Pat. No. 2,846,407 to Wilson, the teachings of which are incorporated by reference herein.

The term, “blood cell” or “hematocyte” or hematopoietic cell” refers to a cell produced through hematopoiesis and is normally found in blood. In mammals, these cells fall into three general categories: red blood cells (erythrocytes), white blood cells (leukocytes), and platelets (thrombocytes). Together, these three kinds of blood cells add up to a total 45% of the blood tissue by volume, with the remaining 55% of the volume composed of plasma, the liquid component of blood. Peripheral blood mononuclear cells (PBMCs) comprise of any blood cell having a round nucleus (as opposed to a lobed nucleus), a lymphocyte or a monocyte. These blood cells are a component in the immune system to fight infection. These cells can be extracted from whole blood using ficoll, a hydrophilic polysaccharide that separates layers of blood, and gradient centrifugation, which will separate the blood into a top layer of plasma, followed by a layer of PBMCs and a bottom fraction of polymorphonuclear cells (such as neutrophils and eosinophils) and erythrocytes. The polymorphonuclear cells can be further isolated by lysing the red blood cells. Exemplary blood cells include erythrocytes, megakaryocytes, monocytes, and granulocytes. Human peripheral blood mononuclear cells (hPBMCs) are human blood cells (e.g., a lymphocyte or a monocyte) with a round nucleus.

As used in this disclosure, the terms “scar tissue” and “scar tissue formation” include any pathological condition resulting from fibrosis, including keloidosis, fibrocystic conditions and joint stiffness. The terms also include post-surgical adhesions or contractures, keloids, hyperplastic or hypertrophic masses formed following trauma, depressed scars from inflammatory responses including acne, wrinkling, cellulite formation, neoplastic fibrosis, and other fibrotic conditions involving fibroblast proliferation and metabolism at a localized area in the body. Such localized area may also be referred to in this disclosure as a site, situs or biological tissue.

As used in this disclosure, the term, “contraction” refers to a phase of wound healing and repair. If contraction continues for too long, it can lead to disfigurement and loss of function. Thus there is a great interest in understanding the biology of wound contraction, which can be modelled in vitro using the collagen gel contraction assay or the dermal equivalent model. Contraction can begin approximately a week after wounding, when fibroblasts have differentiated into myofibroblasts. Contraction can last for several weeks and can continue after the wound is completely re-epithelialized. Wounds can contract at a speed of up to 0.75 mm per day, depending on how loose the tissue in the wounded area is. Contraction usually does not occur symmetrically; rather most wounds have an ‘axis of contraction’ which allows for greater organization and alignment of cells with collagen. At first, contraction occurs without myofibroblast involvement. Later, fibroblasts, stimulated by growth factors, differentiate into myofibroblasts. Myofibroblasts, which are similar to smooth muscle cells, are responsible for contraction, as they contain the same kind of actin as that found in smooth muscle cells.

The term “acute” wound as described in this disclosure may refer to an injury to the skin that occurs suddenly rather than over time. Acute wounds can happen anywhere on the body and vary from superficial scratches to deep wounds damaging blood vessels, nerves, muscles or other body parts. Many actions can cause an acute wound, including: rough surfaces scraping and rubbing against the skin, sharp pointed objects, such as a nail, poking or jabbing into body tissue, sharp edges or blades, such as a knife, cutting the skin cleanly, hard blows by any objects, and tearing the tissue roughly by sheer force. There are two main types of acute wounds: surgical and traumatic. Surgical wounds are incisions made purposefully by a health care professional and are cut precisely, creating clean edges around the wound. Surgical wounds may be closed (with stitches, staples or adhesive) or left open to heal. The healing process for surgical wounds is classified by their potential for infection. A clean surgical wound is considered uncontaminated, and likely made in an operating room or in a sterile procedure environment. A contaminated surgical wound is one that was possibly contaminated with bacteria but is not yet infected. A dirty surgical wound is one with a bacterial infection. Traumatic wounds are injuries to the skin and underlying tissue caused by a force of some nature. They are classified by the object that caused the force (e.g., an abrasion, a puncture, a laceration, or an incision). Abrasions may be rough surface scrapes or rub the skin, causing trauma and tearing the tissue, such as the knee scraping against asphalt. Punctures may occur when a pointed object pokes into the tissue, sometimes causing deep multi-layered trauma, such as the foot stepping on a nail. A laceration may occur when a sharp object delivers a hard blow to the tissue, resulting in a tear that can be jagged and irregular, such as bumping a leg on a table, causing a break in the skin. An incision may occur when an edged cut to the skin is caused by a sharp blade such as cutting a finger with a knife.

The term, “chronic wound,” as described in this disclosure as a wound that does not heal in an orderly set of stages and in a predictable amount of time the way most wounds do; wounds that do not heal within a standard amount of time (e.g., three months) are often considered chronic. Chronic wounds seem to be detained in one or more of the phases of wound healing. For example, chronic wounds often remain in the inflammatory stage for too long. In acute wounds, there is a precise balance between production and degradation of molecules such as collagen; in chronic wounds this balance is lost and degradation plays too large a role.

“Patient,” “subject,” “patient in need thereof,” and “subject in need thereof” are used interchangeably in this disclosure, and refer to a living organism suffering from or prone to a disease or condition that can be treated by administration using the methods and compositions provided in this disclosure. Non-limiting examples include humans, other mammals, bovines, rats, mice, dogs, monkeys, goat, sheep, cows, deer, and other non-mammalian animals. In some embodiments, a patient is human. Tissues, cells and their progeny of a biological entity obtained in vitro or cultured in vitro are also contemplated.

The terms “treat,” “treating” or “treatment,” and other grammatical equivalents as used in this disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. As used in this disclosure, “wound healing” means treating, ameliorating, or improving a condition of a wound involving, without being limiting examples, any one or more of the following the events: (1) rapid hemostasis; (2) appropriate inflammation; (3) mesenchymal cell differentiation, proliferation, and migration to the wound site; (4) suitable angiogenesis; (5) prompt re-epithelialization (re-growth of epithelial tissue over the wound surface); and (6) proper synthesis, cross-linking, and alignment of collagen to provide strength to the healing tissue.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a phenotype or outcome in the absence of a composition as described in this disclosure (including embodiments and examples).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture. In some embodiments contacting includes allowing a composition described in this disclosure to interact with a subject.

Throughout the description and claims of this specification the word “comprise” and other forms of the word, such as “comprising” and “comprises,” means including but not limited to, and is not intended to exclude, for example, other components.

The term “hydration,” or “hydrating” a wound refers to the hydration state of the keratinocytes in the epidermis. Keratinocytes regulate fibroblast behavior through the production of pro- and anti-fibrotic soluble factors, and that the production of those factors is dependent on the hydration state of the keratinocytes.

By “co-administer” it is meant that a composition described in this disclosure is administered at the same time, just prior to, or just after the administration of additional therapies. The composition of the disclosure can be administered alone or can be co-administered to the patient. Co-administration is meant to include simultaneous or sequential administration of the composition individually or in combination (more than one composition or agent). The preparations can also be combined, when desired, with other active substances (e.g. for wound healing).

As used in this disclosure, “sequential administration” includes that the administration of two agents (e.g., compositions described in this disclosure) occurs separately on the same day or do not occur on a same day (e.g., occurs on consecutive days).

As used in this disclosure, “concurrent administration” includes overlapping in duration at least in part. For example, when two agents (e.g., any compositions described in this disclosure that has bioactivity) are administered concurrently, their administration occurs within a certain desired time. The agents' administration may begin and end on the same day. The administration of one agent can also precede the administration of a second agent by day(s) as long as both agents are taken on the same day at least once. Similarly, the administration of one agent can extend beyond the administration of a second agent as long as both agents are taken on the same day at least once. The compositions/agents do not have to be taken at the same time each day to include concurrent administration.

As used in this disclosure, “intermittent administration includes the administration of an agent for a period of time (which can be considered a “first period of administration”), followed by a time during which the agent is not taken or is taken at a lower maintenance dose (which can be considered “off-period”) followed by a period during which the agent is administered again (which can be considered a “second period of administration”). Generally, during the second phase of administration, the dosage level of the agent will match that administered during the first period of administration but can be increased or decreased as medically necessary.

As used in this disclosure, the term “administering” means administration as a suppository, topical contact (e.g., a spray), parenteral, intramuscular, intralesional, intrathecal, intranasal or subcutaneous administration to a subject. Administration is by any route, including parenteral and transmucosal (e.g., buccal, sublingual, palatal, gingival, nasal, vaginal, rectal, or transdermal). Other modes of delivery include, but are not limited to, the use of liposomal formulations, intravenous infusion, transdermal patches, etc.

As used in this disclosure, an “effective amount” or “therapeutically effective amount” is that amount sufficient to affect a desired biological effect, such as beneficial results, including clinical results. As such, an “effective amount” depends upon the context in which it is being applied. An effective amount may vary according to factors known in the art, such as the disease state, age, sex, and weight of the individual being treated. Several divided doses may be administered daily or the dose may be proportionally reduced as indicated by the exigencies of the therapeutic situation. In addition, the compositions/formulations of this disclosure can be administered as frequently as necessary to achieve a therapeutic amount.

A weight percent of a component, unless specifically stated to the contrary, is based on the total weight of the formulation or composition in which the component is included.

The term “about” refers to any minimal alteration in the concentration or amount of an agent that does not change the efficacy of the agent in preparation of a formulation and in treatment of a disease or disorder. The term “about” with respect to concentration range of the agents (e.g., therapeutic/active agents) of the current disclosure also refers to any variation of a stated amount or range which would be an effective amount or range. In embodiments, the term “about” may include ±15% of a specified numerical value or data point.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

### Composition

In one aspect, the present disclosure includes or may be include a composition (e.g., an engineered biomaterial) including extracellular matrix (ECM) components of a mammalian tissue and a polymer. The present disclosure provides a composition (e.g., an engineered biomaterial) including ECM components and a polymer, for use in therapy. The composition of the present disclosure (e.g., an engineered biomaterial) is or may be a gel composition. The mammalian tissue of the composition of the present disclosure may include epithelial and/or connective tissue. The tissue may be obtained from a mammal, e.g., pig, cow, lamb, goat, sheep, or primate (e.g., human). The tissue may be obtained from a pig. The tissue may be autologous or allogenic. The mammalian tissue is or may be a decellularized mammalian tissue. The composition is or may include mammalian tissue and/or extracellular matrix (ECM) components of mammalian tissue, one or more buffers, and one or more of: nutrients, growth factors, and/or polymers. The buffer is or may be a complete cell culture medium. The cell culture medium is or may be serum-free or serum containing medium. The composition is or may include constituents of keratinocyte medium and/or thioglycolate. The nutrients may include one or more of: amino acid, monosaccharide, vitamin, inorganic ion and trace element, and/or salt. The nutrient may be an amino acid. The nutrient may be a monosaccharide. The nutrient may be a vitamin. The nutrient may be an inorganic acid. The nutrient may be a trace element. The nutrient may be a salt. The amino acid included as a nutrient may be one or more of: L-ArginineHCl, L-Cystine2HCl, L-CystineHCl H₂O, L-HistidineHCl H₂O, L-Isoleucine, L-Leucine, L-LysineHCl, L-Methionine, L-Phenylalanine, L-Threonine, L-Tryptophan, L-Tyrosine2H₂O, L-Valine, L-Alanine, L-Asparagine, L-Aspartic acid, L-Glutamic acid, Glycine, L-Proline, L-Serine, and/or L-Hydroxyproline. Vitamine included as a nutrient may be one or more of: K—Ca-Pantothenate, Choline Chloride, Folic acid, i-Inositol, Niacinamide, Pyridoxal HCl, Pyridoxine HCl, Riboflavin, Thiamine HCl, Biotin, Vitamin B12, Para-aminobenzoic acid, Niacin, Ascorbic acid, α-Tocopherol phosphate, Calciferol, Menadione, Vitamin A. Other compounds may be present as a nutrient, for example, one or more of: D-Glucose, Phenol red, HEPES, Sodium pyruvate, Glutathione (reduced), Hypoxantine.Na, Thymidine, Lipoic acid, Putrescine 2HCl, Bacto-peptone, Thymine, Adenine sulphate, Adenosine-5-triphosphate, Cholesterol, 2-deoxy-D-ribose, Adenosine-5-phosphate, Guanine HCl, Ribose, Sodium acetate, Tween 80, Uracil, or Xanthine Na. Inorganic salts added as nutrient may be one or more of: CaCl₂, KCl, MgSO₄, NaCl, NaHCO₃, NaHPO₄, KNO₃, NaSeO₃, Ca(NO₃)₂, CuSO₄, NaHPO₄, MgCl₂, Fe(NO₃)₃, CuSO₄, FeSO₄, Or KH₂PO₄.

The composition of the present disclosure is or may be an engineered biomaterial. The biomaterial is or may include natural biomaterials, e.g., collagen, fibrin, silk, peptides, brush polymers, agarose, alginate, hyaluronic acid, and Chitosan. The biomaterial may be hyaluronic acid. The biomaterial may be collagen. The biomaterial may be silk. The biomaterial may be peptides. The biomaterial may be brush polymers. The biomaterial may be agarose. The biomaterial may be alginate. The biomaterial may be Chitosan. The biomaterial is or may include organic polymers, e.g., one or more of: Poly glycolic acid (PGA), Poly lactic acid (PLA), Poly (lactic-co-glycolic acid) (PLGA), and/or polyethylene glycol (PEG). The biomaterial may be Poly glycolic acid (PGA). The biomaterial may be Poly lactic acid (PLA). The biomaterial may be Poly (lactic-co-glycolic acid) (PLGA). The biomaterial may be polyethylene glycol (PEG). In embodiments, the biomaterial includes inorganic components, e.g., ceramic, metal, and/or hydroxyapatite. The biomaterial is or may include one or more of: collagen, fibrin, silk, peptides, brush polymers, agarose, alginate, hyaluronic acid, Chitosan, PGA, PLA, PLGA, PEG, ceramic, metal, or hydroxyapatite.

The biomaterial of the present disclosure may be in a gel form, sponge form, foam form, patch form, or a semi-liquid/fluid form. The biomaterial may be in gel form.

Extracellular matrix (ECM) is composed of collagens, proteoglycans, structural proteins and basement membrane (e.g., a delicate membrane of protein fibers and glycosaminoglycans separating an epithelium from underlying tissue) and is the largest component of normal skin, which provides the unique properties of elasticity, tensile strength and compressibility of the skin. An important step in the regenerating process is synthesizing the same ECM, resulting in tissue remodelling and less scar formation. In order to augment and support this natural procedure, introduction of a similar template to the wound area may induce efficient cell migration, proliferation, differentiation and generate natural ECM. Non-human mammalian skin, e.g., pig skin, possesses a comparable structure to natural human ECM and the presence of several similar human skin ECM components has been verified in pig skin.

The ECM components include collagen, elastin, and/or sulfated glycosaminoglycans (GAGs). In embodiments, components of the ECM are or may be isolated and/or purified from a tissue of a mammal (e.g., human) or generated using recombinant DNA technology involving gene or gene fragments of a mammal (e.g., human) encoding the respective ECM component (e.g., collagens, elastins, laminins, glycosaminoglycans, proteoglycans, antimicrobials, chemoattractants, cytokines, and growth factors) and expressed in from a suitable expression system (prokaryotic or eukaryotic (e.g., insect or mammalian cells)), and subsequently isolated and/or purified by suitable method in the art. Collagen is one of the most important components of a regenerated skin. In embodiments, increased and organized collagen deposition is associated with improved wound bed maturation.

PBMCs may be included in the composition (e.g., an engineered biomaterial) of the present disclosure. PBMCs may be autologous PBMCs. The pro-angiogenic and regenerative properties of human PBMC allow the composition of the present disclosure (e.g., an engineered biomaterial) with PBMCs to have improved angiogenesis thereby accelerated formation of a mature, well-vascularized wound bed. Adding PBMC to the composition (e.g., an engineered biomaterial) appears to be both safe and feasible with no significant adverse systemic health effects. Seeding uncultured human PBMCs into the dermal composition (e.g., an engineered biomaterial) may have an enhanced tissue integration effect by increasing blood vessel formation, maturation and matrix remodeling. The composition of the present disclosure (e.g., an engineered biomaterial), with or without PBMCs of the present disclosure, regenerates skin after thermal burns. Utilizing uncultured population of cells may facilitate potential clinical application.

The present disclosure includes that expression of collagen in the healing skin biopsies of animals treated with the composition (e.g., an engineered biomaterial) of the present disclosure (e.g., pig skin based gel (PSG)) is abundant. The present disclosure includes that expression of collagen in the healing skin biopsies of animals treated with the composition (e.g., an engineered biomaterial) including human PBMC (hPBMC) (e.g., PSG+hPBMC) is abundant. In embodiments, decellularized mammalian tissue (e.g., skin) may retain extracellular matrix proteins such as collagen and elastin, both of which are important components of the normal tissue (e.g., skin). The higher amounts of collagen in the composition (e.g., an engineered biomaterial (e.g., gel composition)) of the present disclosure may promote more rapid infiltration of host cells to the wound and more prompt wound stabilization.

The tissue from which the ECM component is obtained for use in the composition of the present disclosure is from a mammal. The mammal may be a pig, with a knockout mutant for the α-Gal (Galα1,3-Galβ1-4GlcNAc-R) epitope. The α-Gal (Galα1,3-Galβ1-4GlcNAc-R) epitope, (also a major xenoantigen), is the first barrier in a porcine-to-man tissue and organ xenotransplantation. Skin grafts taken from galactose al, 3 galactose (Gal/gal) knockout pigs are decellularized. The process of decellularization may not result in complete removal of all cellular material.

The composition of the present disclosure (e.g., an engineered biomaterial) is or may include a carbohydrate based polymer. For example, the polymer may be a disaccharide polymer. A disaccharide polymer may be, e.g., hyaluronic acid.

The composition of the present disclosure (e.g., an engineered biomaterial) may include a polymer, where the polymer is a carbohydrate based polymer (e.g., a disaccharide). The disaccharide polymer may include hyaluronic acid. The composition of the present disclosure (e.g., an engineered biomaterial) may include blood cells or platelet rich plasma. The composition of the present disclosure (e.g., an engineered biomaterial) includes peripheral blood mononuclear cells (PBMCs). The PBMCs used in the composition of the present disclosure are or may be autologous human PBMCs.

The composition of the present disclosure includes or may include cells such as, but not limited to, bone marrow-derived stem or progenitor cells, bone marrow mononuclear cells, mesenchymal stem cells (MSC), umbilical cord derived stem cells, mutltipotent adult progenitor cells, whole-blood derived stem or progenitor cells such as endothelial stem cells, endothelial progenitor cells, smooth muscle progenitor cells, whole blood, peripheral blood, and any cell populations that can be isolated from whole blood. The progenitor cells are defined as cells that are committed to differentiate into one type of cells. For example, endothelial progenitor cells means cells that are programmed to differentiate into endothelial cells; smooth muscle progenitor cells means cells that are programmed to differentiate into smooth muscle cells. Progenitor cells in whole blood or peripheral blood includes population of uncommitted and/or committed cells, such as pluripotent cells or totipotent cells.

The composition of the present disclosure may also include a growth factor. The growth factor in the composition of the present disclosure is or may be one or more of: granulocyte macrophase-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C—C motif) ligand 5), IL-8, IGFs, fibroblast growth factor (FGF)-1, FGF-2, FGF-3, FGF-4, FGF-5, FGF-6, FGF-7, FGF-8, FGF-9, transforming growth factor (TGF)-β, VEGF, platelet-derived growth factor (PDGF)-A, PDGF-B, HB-EGF, hepatocyte growth factor (HGF), tumor necrosis factor (TNF)-α, insulin-like growth factor (IGF)-I, and any combination(s) thereof. The growth factor in the composition of the present disclosure is or may be GM-CSF. The growth factor in the composition of the present disclosure is or may be IL-3. The growth factor in the composition of the present disclosure is or may be IL-4. The growth factor in the composition of the present disclosure is or may be NT-6. The growth factor in the composition of the present disclosure is or may be HB-GAM. The growth factor in the composition of the present disclosure is or may be MK. The growth factor in the composition of the present disclosure is or may be IP-10. The growth factor in the composition of the present disclosure is or may be PF-4. The growth factor in the composition of the present disclosure is or may be MCP-1. The growth factor in the composition of the present disclosure is or may be CCL-5. The growth factor in the composition of the present disclosure is or may be IL-8. The growth factor in the composition of the present disclosure is or may be IGFs. The growth factor in the composition of the present disclosure is or may be (FGF)-1, FGF-2, FGF-3, FGF-4, FGF-5, FGF-6, FGF-7, FGF-8, or FGF-9. The growth factor in the composition of the present disclosure is or may be transforming growth factor (TGF)-β. The growth factor in the composition of the present disclosure is or may be VEGF. The growth factor in the composition of the present disclosure is or may be platelet-derived growth factor (PDGF)-A. The growth factor in the composition of the present disclosure is or may be PDGF-B. The growth factor in the composition of the present disclosure is or may be HB-EGF. The growth factor in the composition of the present disclosure is or may be hepatocyte growth factor (HGF). The growth factor in the composition of the present disclosure is or may be tumor necrosis factor (TNF)-a. The growth factor in the composition of the present disclosure is or may be insulin-like growth factor (IGF)-I.

The ECM components in the composition (e.g., an engineered biomaterial) may include collagen, elastin, and/or sulfated glycosaminoglycans (GAGs). The composition (e.g., an engineered biomaterial) may include ribbon-like fibers. The fibers may be about 1 μm-about 40 μm (e.g., about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, or about 40 μm) in width, about 0.1 μm-about 10 μm (e.g., about 0.1 μm, about 0.5 μm, about 1.0 μm, about 1.5 μm, about 2.0 μm, about 2.5 μm, about 3.0 μm, about 3.5 μm, about 4.0 μm, about 4.5 μm, about 5.0 μm, about 5.5 μm, about 6.0 μm, about 6.5 μm, about 7.0 μm, about 7.5 μm, about 8.0 μm, about 8.5 μm, about 9.0 μm, about 9.5 μm, or about 10.0 μm) in thickness, and/or about ≥70 μm-about ≤4000 μm (e.g., about 70 μm, about 75 μm, about 80 μm, about 85 μm, about 90 μm, about 95 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, about 700 μm, about 725 μm, about 750 μm, about 775 μm, about 800 μm, about 825 μm, about 850 μm, about 875 μm, about 900 μm, about 925 μm, about 950 μm, about 975 μm, about 1000 μm, about 1250 μm, about 1500 μm, about 1750 μm, about 2000 μm, about 2250 μm, about 2500 μm, about 2750 μm, about 3000 μm, about 3250 μm, about 3500 μm, about 3750 μm, or about 4000 μm) in length. The fibers may be of about 5 μm-about 30 μm in width. The fibers may be of about 0.5 μm-about 5 μm in thickness. The fibers may be of about ≥75 μm-about ≤800 μm in length. The present disclosure includes all intervening numbers of the ranges indicated.

Optimal wound repair depends on the coordinated contribution of multiple cellular processes (migration, proliferation, collagen synthesis and deposition) that are influenced by inflammatory responses as well as growth factor and cytokine production. The inflammatory process is directly linked to the wound healing response, and inflammatory cytokines stimulate re-epithelization of skin wounds and promote the growth of proliferating keratinocytes. In embodiments, the present disclosure includes a composite biomaterial composed of uncultured PBMC and porcine dermal components for inducing efficient inflammatory responses, which leads to successful and accelerated wound healing.

The present disclosure includes or may include decellularization of xenogeneic tissues to reduce the antigenicity and to retain many of the main components of the extracellular matrix (ECM) components. The present disclosure includes or may include decellularizing mammalian tissue (e.g. skin, placenta, or umbilical cord), to produce a composition for healing wounds. In embodiments, the composition is a biomaterial. The composition of the present disclosure is or may be processed to produce a gel. The gel may be a pig skin gel (PSG) prepared by the method described in the present disclosure. In the present disclosure, the feasibility and effectiveness of this porcine-obtained ECM for wound healing is investigated in a nude-mouse full-thickness cutaneous wound model. Embedding human peripheral blood mononuclear cells (hPBMC) in the gel (e.g., PSG) in the method of preparing the composition of the present disclosure further accelerated formation and maturation of well-organized wound tissue in the setting of acute skin wounds.

A pig skin-obtained ECM gel composed of various ECM components and endogenous growth factors is developed as a biomaterial for skin tissue engineering. The ECM gel is prepared from decellularized, homogenized, and lyophilized skin of a Gal/gal knockout pig. The composition of the present disclosure is prepared for direct application on a wound with or without human cell addition. The ECM gel composition of the present disclosure contributes to the wound healing rates within 15 days, and interact and protect the wound in a subject in need thereof by providing good adherence of cells and a favorable (e.g., moist) healing environment. The addition of human cells to the gel prior to wound application, markedly improves host blood vessel formation in the wounds and significantly accelerate the wound healing process. The healing is or may be achieved within 4, 5, 6, 7, 8, 9, 10, 11, 12, 13, 14 or 15 days of applying the composition of the present disclosure to the wound. The healing of a wound, for example, skin tear, friction, closed impact surgical wound, skin abrasion, burn (first-degree, second-degree, and/or third-degree), skin incision, skin laceration, skin contusion, skin puncture, pressure ulcer, venous ulcers, arterial ulcers, neuropathic/diabetic wounds, lymphedema, or surgical site incision, is achieved within 4, 5, 6, 7, 8, 9, 10, 11, 12, 13, 14 or 15 days of applying the composition of the present disclosure to the wound.

In the same group of animals, human blood vessels are found in the wounds at days 5 and 10 as evidenced by the expression of human CD31, and positive staining with the human specific anti-mitochondrial antibody. Furthermore, the qPCR detection system confirmed the presence of low numbers of human cells; however, identification of the few human specific cells in a high background of other contaminating host (mouse) cells was difficult. The present disclosure includes a biomaterial including human blood cells (e.g., hPBMCs), results in survival of the human cells and neovascularization of the wound. The biomaterial including hPBMCs accelerated wound healing.

Human peripheral blood cells and ECM components of decellularized mammalian tissue and a polymer in the biomaterial of the present disclosure, promotes migration of keratinocytes and epithelial cells, as well as neovascularization due to compositional properties, leading to improved wound healing.

### Methods of Treatment or Use

In one aspect, the present disclosure provides a method of wound healing in a subject, the method including applying a composition (e.g., an engineered biomaterial) including ECM components of a mammalian tissue and a polymer to a wound of the subject. In another aspect, the present disclosure provides a method of delivering a therapeutic agent to a wound of a subject in need thereof, the method including applying a composition (e.g., an engineered biomaterial) including extracellular matrix (ECM) components of a mammalian tissue, a polymer, and a therapeutic agent to a wound of the subject. The mammalian tissue may be a decellularized mammalian tissue. The present disclosure provides that the addition of human cells to the gel prior to wound application, markedly improves host blood vessel formation in the wounds and significantly accelerate the wound healing process. The healing is or may be achieved within 4, 5, 6, 7, 8, 9, 10, 11, 12, 13, 14 or 15 days of applying the composition of the present disclosure to the wound. The healing of a wound, for example, skin tear, friction, closed impact surgical wound, skin abrasion, burn (first-degree, second-degree, and/or third-degree), skin incision, skin laceration, skin contusion, skin puncture, pressure ulcer, venous ulcers, arterial ulcers, neuropathic/diabetic wounds, lymphedema, or surgical site incision, is achieved within 4, 5, 6, 7, 8, 9, 10, 11, 12, 13, 14 or 15 days of applying the composition of the present disclosure to the wound. The wound may be a skin tear. The wound may be friction. The wound may be a closed impact surgical wound. The wound may be a skin laceration. The wound may be a skin contusion. The wound may be a skin puncture. The wound may be a pressure ulcer. The wound may be venous ulcers. The wound may be arterial ulcers. The wound may be neuropathic/diabetic wound. The wound may be lymphedema. The wound may be a surgical site incision.

In further aspect, the present disclosure provides a method of delivering a growth factor to a wound of a subject in need thereof, the method including applying a composition (e.g., an engineered biomaterial) including extracellular matrix (ECM) components of a mammalian tissue, a polymer, and a growth factor to a wound of the subject. The mammalian tissue may be a decellularized mammalian tissue. The growth factor used in the method of the present disclosure is one or more of: granulocyte macrophase-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C—C motif) ligand 5), IL-8, IGFs, fibroblast growth factor (FGF)-1, FGF-2, FGF-3, FGF-4, FGF-5, FGF-6, FGF-7, FGF-8, FGF-9, transforming growth factor (TGF)-β, VEGF, platelet-derived growth factor (PDGF)-A, PDGF-B, HB-EGF, hepatocyte growth factor (HGF), tumor necrosis factor (TNF)-α, insulin-like growth factor (IGF)-I, or any combination(s) thereof. The growth factor used in the method of the present disclosure is or may be GM-CSF. The growth factor used in the method of the present disclosure is or may be IL-3. The growth factor used in the method of the present disclosure is or may be IL-4. The growth factor in the composition of the present disclosure is or may be NT-6. The growth factor used in the method of the present disclosure is or may be HB-GAM. The growth factor used in the method of the present disclosure is or may be MK. The growth factor used in the method of the present disclosure is or may be IP-10. The growth factor used in the method of the present disclosure is or may be PF-4. The growth factor used in the method of the present disclosure is or may be MCP-1. The growth factor used in the method of the present disclosure is or may be CCL-5. The growth factor used in the method of the present disclosure is or may be IL-8. The growth factor used in the method of the present disclosure is or may be IGFs. The growth factor used in the method of the present disclosure is or may be (FGF)-1, FGF-2, FGF-3, FGF-4, FGF-5, FGF-6, FGF-7, FGF-8, or FGF-9. The growth factor used in the method of the present disclosure is or may be transforming growth factor (TGF)-β. The growth factor used in the method of the present disclosure is or may be VEGF. The growth factor used in the method of the present disclosure is or may be platelet-derived growth factor (PDGF)-A. The growth factor used in the method of the present disclosure is or may be PDGF-B. The growth factor used in the method of the present disclosure is or may be HB-EGF. The growth factor used in the method of the present disclosure is or may be hepatocyte growth factor (HGF). The growth factor used in the method of the present disclosure is or may be tumor necrosis factor (TNF)-α. The growth factor used in the method of the present disclosure is or may be insulin-like growth factor (IGF)-I.

In yet another aspect, the present disclosure provides a method of hydrating a wound of a subject in need thereof, the method including applying a composition (e.g., an engineered biomaterial) including extracellular matrix (ECM) components of a mammalian tissue and a polymer to a wound of the subject. The mammalian tissue may be a decellularized mammalian tissue.

The composition used in the method of wound healing or delivering a therapeutic agent to a wound in disclosed in the previous section of the present disclosure, and is incorporate by reference in its entirety in this section.

Wound healing is the process in which cells in the body regenerate and repair to reduce size of damaged or necrotic area. Wound healing involves a set of phases that include inflammation surrounding a region of injury, cell migration and mitosis, angiogenesis and the development of granulation tissue, repair of the connective tissue, regeneration of extracellular matrix and remodelling that leads to a healed wound. Each phase has its distinctive mechanism and is also interconnected with the other phases. The duration of these individual phases varies depending on the intensity and depth of the wound. Most wound dressing treatments aim to facilitate these stages of wound healing by providing a moist environment, controlling excessive exudate buildup, and protecting against infection that would perturb normal healing.

Skin injuries caused by various physical factors or chemical agents can induce wound healing and skin regeneration. However, in the therapy of extensively burned patients, the limited amount of donor sites for skin auto transplantation is a constant problem. While full-thickness wounds are resurfaced with split-thickness skin autografts, deep dermal burns are usually covered with biological or synthetic covers (dressings). Wound covers work as temporary substitutes. If the wound does not heal spontaneously, they have to be replaced with the patient's own skin.

The present disclosure provides a method of healing wounds. Wounds may be classified by several methods, including their cause, location, type of injury (or symptoms), wound depth, and tissue loss or clinical appearance of the wound. General wounds are classified as being superficial (loss of the epidermis only), partial thickness (both the epidermis and dermis are affected), and full thickness (the dermis, subcutaneous fat and sometimes bone are affected). A partial thickness wound may appear pink, is often painful, and no yellow tissue is observed. Full thickness wounds may involve damage or impairment to all layers of the skin, as well as underlying subcutaneous tissue and deeper tissues, including bone, muscle, tendon, nerve tissue, vascular tissue, or visceral organs at the site of injury.

A full thickness wound or injury may be caused by at least one of the following: a blunt force trauma; a penetrating trauma; a gunshot wound; a microbial infection; a necrotizing infection; a bacterial infection; a fungal infection; hypothermia; frostbite; ischemia; tissue hypoxia; reperfusion of ischemic tissue; microvascular disease; a vascular disease associated with diabetes; non-mobility or immobility; gangrene; sepsis; septic shock; osteomyelitis; cellulitis; vasculitis; diabetes mellitus; diabetic ulcer; diabetic foot ulcer; cancer; leukemia; cirrhosis; chronic fibrosis; atherosclerosis; edema; sickle cell disease; arterial insufficiency-related illnesses; immune suppression; use of an immunosuppressive drug; use of a chemotherapeutic drug; use of a steroid; exposure to extreme temperature; exposure to a biological toxin; exposure to a poison; a snakebite; an insect bite; an insect sting; a sting from a poisonous fish; or from a sting from a jellyfish. Moreover, the subject may develop or may be at risk of developing at least one of the following conditions: sepsis, septic shock, a microbial infection, a necrotizing infection, necrotizing fasciitis, gangrene, or osteomyelitis.

Full thickness wound or injury may be caused by or is associated with an infection, for example, one or more of: crepitant anaerobic cellulitis; necrotizing fasciitis; nonclostridial myonecrosis; clostridial myonecrosis; fungal necrotizing cellulitis; gonococcal arthritis; nongonacoccal arthritis; bacterial arthritis; granulomatous arthritis; hemotogenous osteomyelitis; contiguous-focus osteomylitis; chronic osteomyelitis; bacterial osteomyelitis; fungal osteomyelitis; and the like. Exemplary organisms which may cause such infections include one or more of the following: Bacteroides species, Peptostreptococcus species, Clostridium species, members of the family Enterobacteriaceae, Fusobacterium species, Streptococcus pyogenes, Staphylococcus aureus, Streptococcus agalactiae, Clostridium perfringens, Clostridium novyi, Clostridium septicum, Clostridium histolyticum, Clostridium fallax, Clostridium bifermentans, Phycomyces species, Aspergillus species, Rhizopus species, Mucor species, Absidia species, Neisseria gonorrhoeae, Escherichia coli, Shigella species, Salmonella species, Campylobacter species, Yersinia species, Streptobacillus moniliformis, Haemophilus influenzae, Mycobacterium tuberculosis, Blastomyces species, Cryptococcus species, Sporothrix species, Sporothrix schenckii, Candida species, Pseudomonas aeruginosa.

The present disclosure includes a method of wound healing in a subject, which reduces scar tissue formation. The method of the present disclosure reduces scar tissue formation compared to a wound treated with a composition of hyaluronic acid (HA) but lacking the composition (e.g., an engineered biomaterial) of the present disclosure.

The present disclosure includes a method of wound healing in a subject, which reduces decoloration of the healing wound. The method of the present disclosure reduces decoloration compared to a wound treated with a composition of hyaluronic acid (HA) but lacking the composition (e.g., an engineered biomaterial) of the present disclosure.

The method of wound healing of the present disclosure promotes migration of keratinocytes and epithelial cells. The method of wound healing of the present disclosure promotes neovascularization at the wound. The method of wound healing of the present disclosure promotes neovascularization in about 5 to 10 days after applying the biomaterial to the wound. In the method of wound healing of the present disclosure, neovascularization results in ECM remodeling at the wound. In the method of wound healing of the present disclosure reduced elongated scar is observed at the site of the healed wound, compared to a wound treated with a composition of hyaluronic acid (HA) but lacking the biomaterial of the present disclosure. The biomaterial generates epidermal cells and restores bilayer structure of the epidermis and dermis. The wound may be healed within 15-40 days of applying the biomaterial to the wound. The wound may be healed within 25 days of applying the biomaterial to the wound.

Wound Healing in Combination with a Second Agent

In one aspect, the present disclosure provides wound healing with the composition (e.g., an engineered biomaterial) in this disclosure in combination with a therapeutic agent. In one aspect, the present disclosure includes a composition (e.g., an engineered biomaterial) including extracellular matrix (ECM) components of a mammalian tissue, a polymer, and a pharmaceutical composition of a wound healing or ameliorating agent with an effective dose of the agent. The mammalian tissue may be a decellularized mammalian tissue. The therapeutic agent may be administered as a pharmaceutical composition independent of the wound healing composition (e.g., an engineered biomaterial), by co-administering or concurrently administering, or sequentially administering with the composition (e.g., an engineered biomaterial). In such instances the present disclosure includes that a therapeutic agent may be administered orally, topically, intravenously, intraperitoneally, nasally, or by inhalation. The pharmaceutical composition of the therapeutic agent of the present disclosure may include a pharmaceutically acceptable excipient.

The second therapeutic agent may include a pruritus agent (an anti-itch agent), analgesic agents (e.g., pain relief agents), antiseptic agents, and antibiotics. The therapeutic agent in the pharmaceutical composition of the present disclosure is or may be a wound healing agent/molecule. Such agent/molecule may be suitably present in amounts that are effective for the purpose intended.

An anti-itch agent may include, but is not limited to willow bark extract, salicylic acid, antihistamines (diphenhydramine), corticosteroids (e.g., 1% hydrocortisone cream), benzocaine topical treatment, mint oil, methanol, camphor, ammonium hydroxide, benzyl alcohol, pramixine hydrochloride.

Exemplary analgesic agents can include, but are not limited to, non-narcotics (e.g., acetaminophen), non-steroidal anti-inflammatory drugs (NSAIDs) (e.g., aspirin, diclofenac, dexibuprofen, diflunisal, etodolac, fenoprofen, flufenamic acid, flurbiprofen, ibuprofen, indomethacin, ketoprofen, ketorolac, lornoxicam, lxoprofen, melofenamic acid, mefenamic acid, meloxicam, nabumetne, naproxen, oxaprozin, piroxicam, salsalate, sulindac, tenoxicam, tolmetin, and tolfenamic acid. Other analgesics include COX-2 inhibitors (e.g., celecoxib, rofecoxib, valdecoxib and etoricoxib), and opioids (e.g., buprenorphine, butorphanol, codeine, hydrocodone, hydromorphone, levophanol, mederidine, methadone, morphine, nalbuphine, oxycodone, oxymorphone, pentazocine, and propoxyphene).

The antiseptic agents may include, but are not limited to, alcohol (e.g., ethanol, 1-propanol, and 2-propanol/isopropanol), quaternary ammonium compounds (benzalkonium chloride, cetyl trimethylammonium bromide, cetylpyridinum chloride, benzethonium chloride), boric acid, brilliant green, chlorhexidine gluconate, and hydrogen peroxide (alone or in combination with acetic acid to make peracetic acid). Other agents may include iodine, Manuka honey, mercurochrome, octenidine dihydrochloride, phenol, sodium chloride, sodium hypochlorite, calcium hypochlorite and balsam of Peru.

The antibiotic agent may include, but are not limited to one or more of: Gentamicin, Kanamycin, Neomycin, Netilmicin, Tobramycin, Paromomycin, Streptomycin, Rifaximin, Loracarbef, Ertapenem, Doripenem, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Vancomycin, Telavancin, Dalbavancin, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfamethizole, Sulfamethoxazole, Sulfasalazine, Sulfisoxazole, Demeclocycline, Doxycycline, Minocycline, Oxytetracycline, Tetracycline, Clofazimine, Isoniazid, Pyrazinamide, Rifabutin, Rifapentine, Streptomycin, Mupirocin, Platensimycin, Quinupristin, Thiamphenicol, and/or Tinidazole.

The effective dose may be between about 0.001 mg/kg to about 100 mg/kg of the agent.

The therapeutic agent may be administered to a subject in need thereof, at a dose between about 0.001 mg/kg to about 0.01 mg/kg of the compound, between about 0.01 mg/kg to about 0.1 mg/kg of the compound, between about 0.1 mg/kg to about 1.0 mg/kg of the compound, between about 1.0 mg/kg to about 5.0 mg/kg of the compound, between about 5.0 mg/kg to about 10 mg/kg of the compound, between about 10 mg/kg to about 15 mg/kg of the compound, between about 15 mg/kg to about 20 mg/kg of the compound, between about 20 mg/kg to about 25 mg/kg of the compound, between about 25 mg/kg to about 30 mg/kg of the compound, between about 30 mg/kg to about 35 mg/kg of the compound, between about 35 mg/kg to about 40 mg/kg of the compound, between about 40 mg/kg to about 45 mg/kg of the compound, between about 45 mg/kg to about 50 mg/kg of the compound, between about 50 mg/kg to about 55 mg/kg of the compound, between about 55 mg/kg to about 60 mg/kg of the compound, between about 60 mg/kg to about 65 mg/kg of the compound, between about 65 mg/kg to about 70 mg/kg of the compound, between about 70 mg/kg to about 75 mg/kg of the compound, between about 75 mg/kg to about 80 mg/kg of the compound, between about 80 mg/kg to about 85 mg/kg of the compound, between about 85 mg/kg to about 90 mg/kg of the compound, between about 90 mg/kg to about 95 mg/kg of the compound, or between about 95 mg/kg to about 100 mg/kg of the compound.

The present disclosure of the present disclosure includes compositions with an effective dose of a composition in which the therapeutic agent may be between about 0.1% to about 20% w/v of the composition.

For example, the effective dose of a therapeutic agent may be between about 0.001%-about 0.01%, between about 0.01%-about 0.1%, between about 0.1%-about 1.0%, between about 1.0%-about 2.0%, between about 2.0%-about 3.0%, between about 3.0%-about 4.0%, between about 4.0%-about 5.0%, between about 5.0%-about 6.0%, between about 6.0%-about 7.0%, between about 7.0%-about 8.0%, between about 8.0%-about 9.0%, between about 9.0%-about 10%, between about 10%-about 11%, between about 11%-about 12%, between about 12%-about 13%, between about 13%-about 14%, between about 14%-about 15%, between about 15%-about 16%, between about 16%-about 17%, between about 17%-about 18%, between about 18%-about 19%, or between about 19%-about 20% w/v of the composition.

### Method of Preparing Composition

In one aspect, the present disclosure includes a method of preparing a composition including extracellular matrix (ECM) components of a mammalian tissue and a polymer, the method including decellularzing the mammalian tissue to prepare a powder including extracellular matrix (ECM) components, mixing the powder with a medium comprising buffer, nutrients, growth factor, polymer, where the powder soaks up the medium, thereby preparing the composition. The mammalian tissue may be a decellularized mammalian tissue. In embodiment, components of the ECM are or may be isolated and/or purified from a tissue of a mammal (e.g., human) or generated using recombinant DNA technology involving gene or gene fragments of a mammal (e.g., human) encoding the respective ECM component (e.g., collagens, elastins, laminins, glycosaminoglycans, proteoglycans, antimicrobials, chemoattractants, cytokines, and growth factors) and expressed in from a suitable expression system (prokaryotic or eukaryotic (e.g., insect or mammalian cells)), and subsequently isolated and/or purified by suitable method in the art.

In the present disclosure the powder for preparing the composition of the present disclosure is prepared by treating the tissue with a chemical, freeze-drying the chemical treated tissue, and homogenization of freeze-dried tissue. In embodiments, the powder is filamentous. The medium is serum containing, reduced serum, or non-serum containing medium. The growth factor is one or more of: Recombinant 4-1BBL, Recombinant 6Ckine, 6Ckine Recombinant Human Protein, ANGPT2 (ANG2), ANGPTL5, Activin A, Activin Rib, BAFF, BAMBI, CXCL13, BDNF, BLC, BMP2, BMP4, BMP5, BMP7, BMPR1A, CCL1, CCL17, CCL20 (MIP-3), CCL21, CD40, GM-CSF, IL-3, IL-4, NT-6, HB-GAM, MK, IP-10, PF-4, MCP-1, RANTES, IL-8, IGFs, FGF 1, FGF-2, FGF-3, FGF-4, FGF-5, FGF-6, FGF-7, FGF-8, FGF-9, TGF-β, VEGF. PDGF-A, PDGF-B, HB-EGF, HGF, TNF-α, IGF-I, or any combination(s) thereof. The nutrients may include amino acid, monosaccharide, vitamin, inorganic ion and trace element, and/or salt. The amino acid included as a nutrient may be one or more of: L-ArginineHCl, L-Cystine2HCl, L-CystineHCl H₂O, L-HistidineHCl H₂O, L-Isoleucine, L-Leucine, L-LysineHCl, L-Methionine, L-Phenylalanine, L-Threonine, L-Tryptophan, L-Tyrosine2H₂O, L-Valine, L-Alanine, L-Asparagine, L-Aspartic acid, L-Glutamic acid, Glycine, L-Proline, L-Serine, and/or L-Hydroxyproline. Vitamine included as a nutrient may be one or more of: K—Ca-Pantothenate, Choline Chloride, Folic acid, i-Inositol, Niacinamide, Pyridoxal HCl, Pyridoxine HCl, Riboflavin, Thiamine HCl, Biotin, Vitamin B12, Para-aminobenzoic acid, Niacin, Ascorbic acid, a-Tocopherol phosphate, Calciferol, Menadione, Vitamin A. Other compounds may be present as a nutrient, for example, one or more of: D-Glucose, Phenol red, HEPES, Sodium pyruvate, Glutathione (reduced), Hypoxantine.Na, Thymidine, Lipoic acid, Putrescine 2HCl, Bacto-peptone, Thymine, Adenine sulphate, Adenosine-5-triphosphate, Cholesterol, 2-deoxy-D-ribose, Adenosine-5-phosphate, Guanine HCl, Ribose, Sodium acetate, Tween 80, Uracil, and/or Xanthine Na. Inorganic salts added as nutrient may be one or more of: CaCl₂, KCl, MgSO₄, NaCl, NaHCO₃, NaHPO₄, KNO₃, NaSeO₃, Ca(NO₃)₂, CuSO₄, NaHPO₄, MgCl₂, Fe(NO₃)₃, CuSO₄, FeSO₄, and/or KH₂PO₄.

### EXAMPLES

### Example 1: Preparation of Porcine Skin Scaffolds

Gal/gal porcine skin was purchased from Avantea (Cremona, Italy). A piece of skin measuring 20*15 cm was dissected from a Gal/gal knock out pig and was vacuum sealed in plastic bag and stored frozen at −200° C. until use. The skin was thawed at room temperature, and cleaned by excision of the subdermal fat tissue, removal of hair and lastly washed with distilled water. The skin was then placed in a 5 L plastic container and agitated at 200 RPM with 0.5% SDS (Sodium Dodecyl Sulfate) (Sigma, Germany) containing 0.02% sodium azide (Sigma, Germany) and 1.86% EDTA (Alfa Aesar, Germany) at 37° C. for 9 days continuously. SDS was changed first after 24 hours and then every 48 hours. During every change of SDS, the skin was washed for an hour with distilled water.

### Decellularized Pig Skin

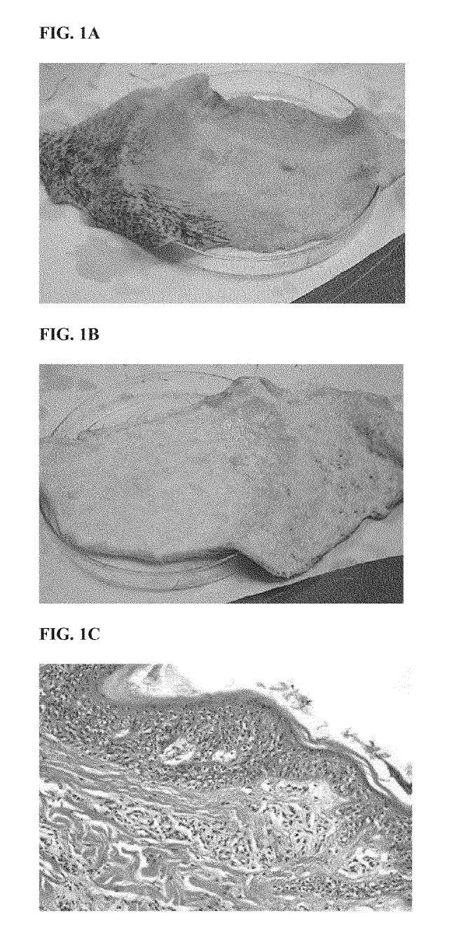

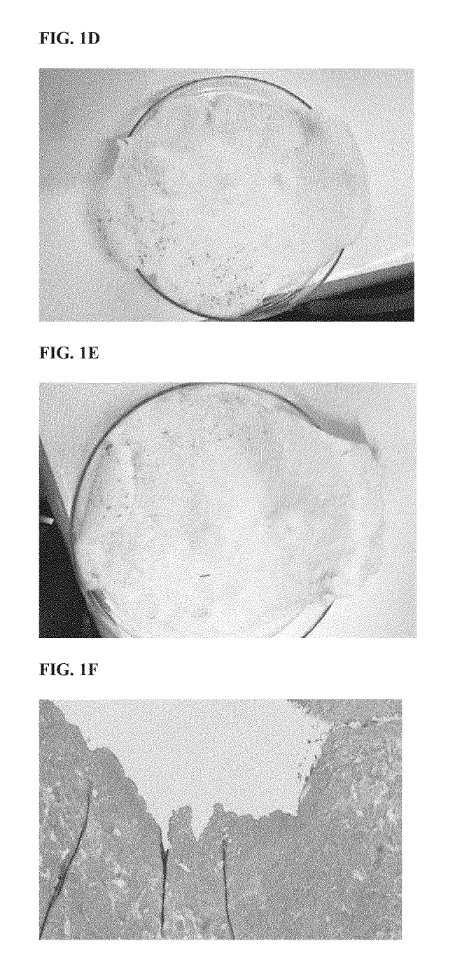

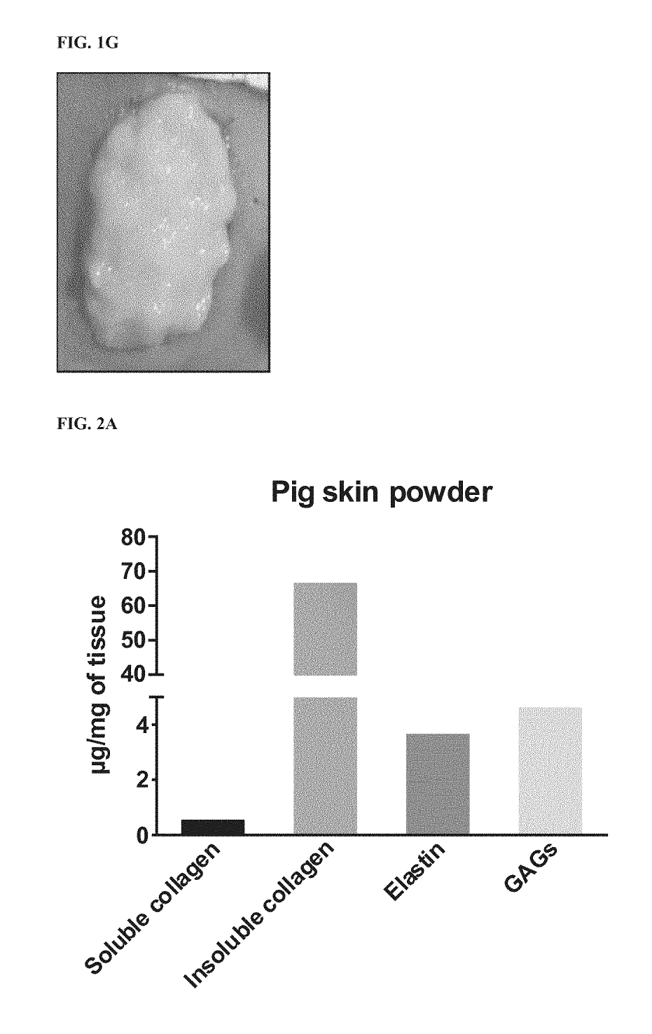

Gal/gal KO pig skin was completely decellularized in 9 days after continuous treatment with 0.5% SDS. The gross morphology and histology of the pig skin before (FIGS. 1A-1C) and after decellularization (FIGS. 1D-1F) are shown in FIGS. 1A-1G. No presence of nuclei (blue) or cellular remnants was observed in the decellularized pig skin (FIG. 1F). The pig skin gel prepared using HA and keratinocyte medium was white in color (FIG. 1G) and was used for all wound skin healing experiments.

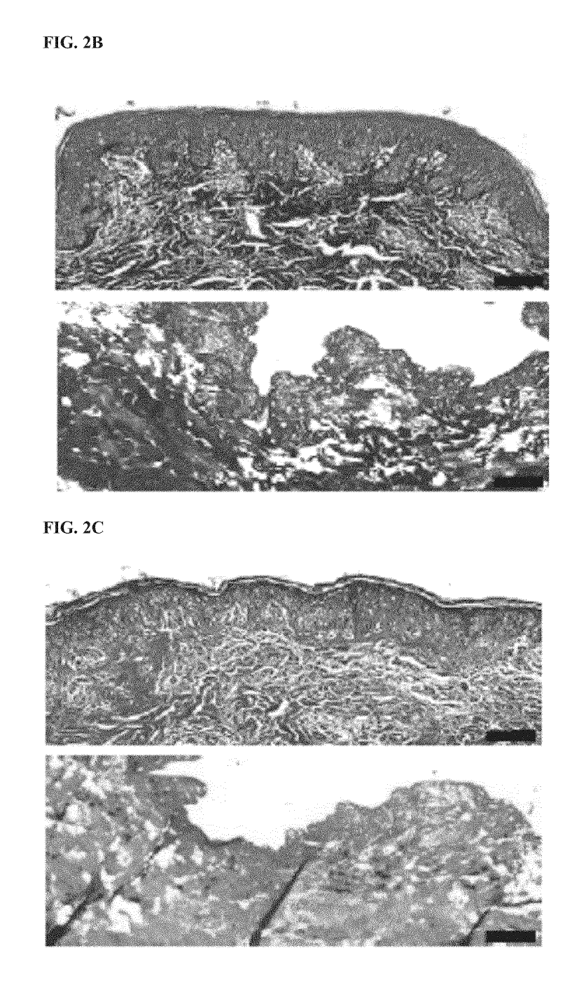

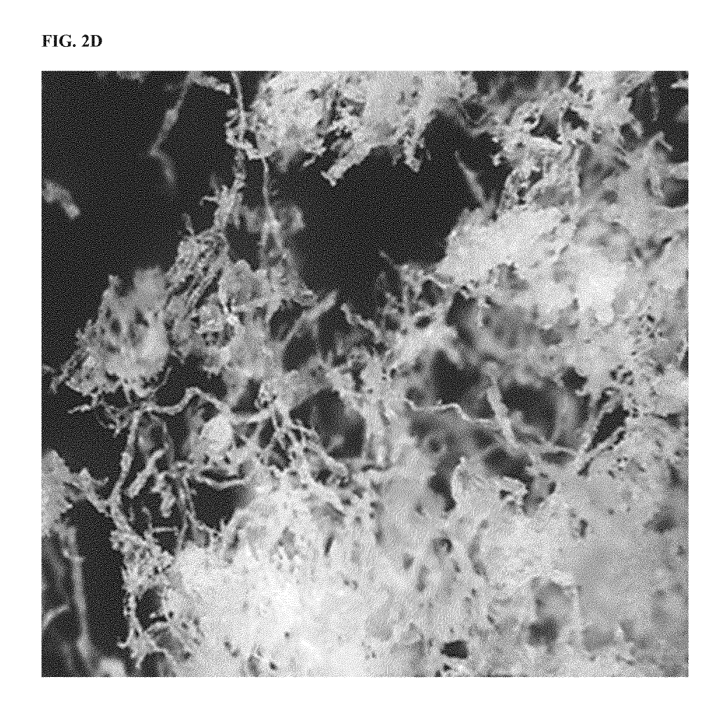

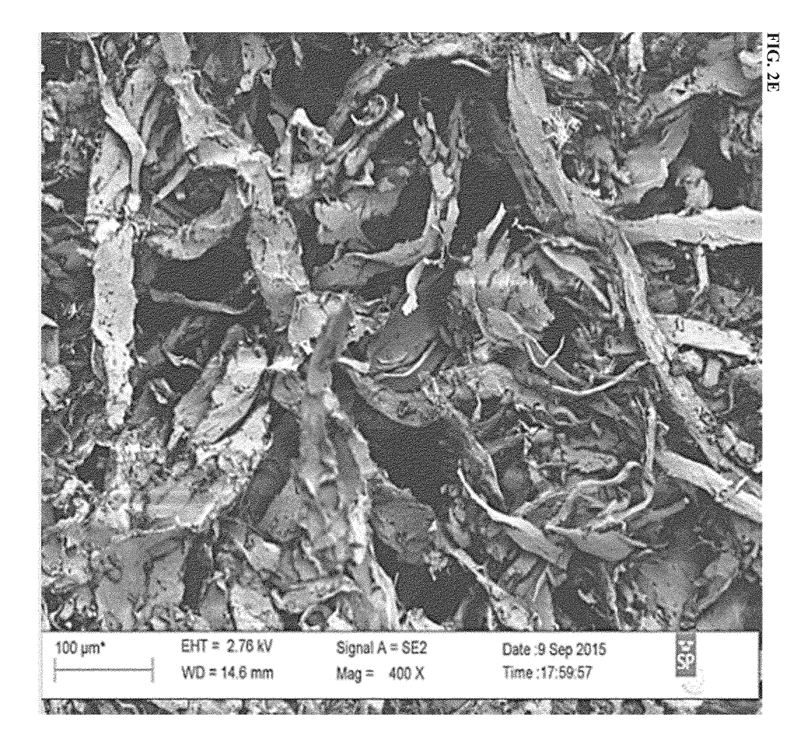

The decellularized pig skin powder retained one of the major ECM components required for regeneration of skin-collagen (66.545 μg/mg), as well as elastin (3.598 μg/mg) and sulfated GAGs (4.555 μg/mg) (FIG. 2A). MT and VVG staining also confirmed that collagen and elastin were preserved after decellularization (FIGS. 2B and 2C). Furthermore, characterization of the powdered pig skin showed that the decellularized pig skin powder had a filamentous, white macroscopic appearance and was transparent to light when investigated by optical microscopy (FIG. 2D). Scanning electron microscopy showed that it consisted of ribbon-like fibers that varied in shape and size. The predominant fiber dimensions were 20-30 μm in width, 1-3 μm in thickness and up to 2 mm in length. The fibers were irregularly wrinkled and twisted, forming easily dispersible bundles (FIGS. 2E and 2F). The sterility testing of the pig skin powder also showed no increase in optical density measured for 2 weeks during culture showing that gamma irradiation can be used for sterilization of pig skin powder.

Verification of Decellularization and Characterization of the Extracellular Matrix

Decellularization was verified using histology and DNA quantification. Two pieces from normal and decellularized skin were fixed in formalin for 48 hours and embedded in paraffin. The sections were cut at 5 μm thickness using a microtome and stained by Hematoxilin Eosin (HE), Masson's Trichrome (MT) (25088, Polysciences, USA) and Verhoeff Van Gieson (VVG) (25089, Polysciences, USA) methods to identify the presence of nuclei, collagen and elastin. 25 mg pieces of tissue were cut from normal and decellularized skin and total DNA was isolated using Qiagen Blood and Tissue DNA kit (Qiagen, Sweden) following the manufacturer's instruction and quantified using nanodrop at a 260 nm wavelength.

### Preparation and Characterization of Decellularized Pig Skin Powder

After decellularization, the skin was washed for 5 days in distilled water. The water was changed twice every 12 hours. The decellularized skin was cut into pieces of 3*3 sq cm and freeze dried (lyophilized) for 72 hours. The lyophilized pieces were pulverized in a cryomill with a 0.75 μm sieve at 14000 rpm. The filamentous powder obtained was sterilized by gamma irradiation at 25 kGy for 3 min and 25 sec.

Extracellular matrix quantification in the pig skin powder for collagen, elastin and glycoaminoglycans (GAG) was performed. The powder structure and morphology was investigated by optical and scanning electron microscopy (SEM). Optical imaging was performed by an Olympus SZX16 stereo microscope equipped by ColorView IIIu CCD camera (Soft Imaging System GmbH, Germany) and Olympus Cell̂D image-analysis software. SEM analysis was performed by a Zeiss Supra 40VP instrument in secondary electron detection mode. To reduce sample charging during SEM imaging, powder samples were deposited on carbon pads and sputter-coated by 15 nm thick Au/Pd film in a Gatan PECS Mod 682 instrument.

### Preparation of Pig Skin Gel (PSG)

A gel was prepared by mixing 50 mg of decellularized skin powder and 250 μl of hyaluronic acid (HA) (Sigma, Germany) to get a gel like consistency. The hyaluronic acid was constituted at 1 mg/ml in keratinocyte medium (Lonza, Calif.).

### Sterility

Three random samples of 1 mg irradiated powder was added into thioglycolate broth (Fluka, USA) and cultured for 2 weeks in an incubator at 37° C. Every other day, 200 μl of broth was collected and verified for turbidity by measuring optical density in spectrophotometer (Synergy 2, Biotech, USA) at 600 nm wavelength. An increase in turbidity indicated contamination.

### Animal Experiments

In total, 72 BALB/c nude female mice, 7-8 weeks of age and weighing 17-18 g (Taconic, Denmark) were used to study wound healing rate. The mice were divided into four groups after induction of full thickness skin wound; i) untreated; ii) treated with HA; iii) treated with PSG only; and iv) treated with PSG+hPBMC (1*10⁶ cells). The mice were anesthetized using isofluorane and Karprofen (Rimadyl) at 10 mg/kg (Pfizer, Luxemborg) was given pre-operative i.p. for post-operative pain relief as well as once daily for 2 days after surgery. The skin was wiped with a few drops of chemical gasoline and a full-thickness 1×1 cm square excision wound was created in the upper back area of each animal. Gel with or without cells was applied using a sterile wooden stick. Tegaderm (3M™, Germany) was then placed on the wounds and sutured to the skin using a 4-0 non-absorbable monofilament suture (Ethicon, Germany) for protection and to keep the gel in place. The tegaderm was further covered with hydrofilm to prevent removal of the tegaderm by the animals. On the day of surgery, peripheral blood was collected from a healthy human donor and mononuclear cells were separated on lymphoprep and the isolated cells were washed in PBS thrice and suspended in keratinocyte medium. The cells were counted using Biorad automated cell counter. At 5, 10, 15 and 25 days after treatment, animals were sacrificed, and the skins, including the wounds, were excised for histological examination. The cutaneous wounds were also photographed.

### Histology and Immunohistochemistry of Wounds

The paraffin blocks of the skin wounds from all groups were sectioned at 5 μm thickness using a microtome and 5 sections, covering the whole region of the wound, were stained with HE, MT and VVG following standard procedure. The slides were scanned using a Leica SCN400 microscope. Skin sections were stained with human specific anti-mitichondria antibody 1-100 (Merck Millipore, Stockholm, Sweden) for detection of human cells. The secondary antibody used was a peroxidase affinipure (Fab)₂ fragment goat-anti-mouse IgG, F(ab)₂ fragment specific 1:500 (Jackson Immunoresearch, West Grove, USA).

### Immunofluorescence

Cryosections were cut at 5 μm thickness using a cryotome. Antibodies specific for human CD31 (1:25; 10148-MM13, Sino Biologicals) and mouse-specific CD31 (1:400, LSC 348704, LSBio) and the secondary antibodies Alexa GαM 568 (1:100; A11031, Life Technologies) and GαR FITC (1:100; SC3825, Santa Cruz) were used. Briefly, cryoslides were fixed in acetone:methanol (4:6) for 10 min at −20° C. and washed in PBS for 5 min. The slides were blocked with serum of secondary antibody host and incubated with primary antibody overnight at +4° C. The slides were washed in PBS thrice and incubated with secondary antibody in dark at +4′C for 45 min. The slides were washed thrice in PBS and cover slipped with a drop of mounting medium containing DAPI (H1500, Vector Laboratories).

### Quantification of Collagen

The amount of collagen in all animal groups on day 25 (newly formed skin) was quantified based on the intensity of blue stained collagen in MT staining using the principle of densitometry. Five representative images were taken from each animal and the total intensity of blue color per image was quantified with BioPix iQ 2.1.8 software. The collagen intensity was read as arbitrary units (a.u) and the average for each animal was calculated.

### Measurement of Wound Area

To calculate the wound size, a measuring scale was placed at the site of the wound and all mice were photographed using a Sony a 35 digital camera. The area of unclosed wound (not scars) per animal was then calculated. The average of unclosed wound area/animal per group is presented.

### Gene Analysis

RNA was extracted by adding 1 ml ice cold Qiazol (Qiagen GmbH) to the frozen skin samples. A steel bead was added to each sample and samples were homogenized in a Qiagen TissueLyser at 25 Hz for 2×5 min, 200 μl chloroform was added and the samples were shaken vigorously for 15s, left at room temperature for 3 min and centrifuged at 12,000 g for 15 min at 4° C. Following centrifugation 350 μl of the supernatant was transferred to a new tube and mixed with 350 μl 70% ethanol and mixed carefully by pipetting and added to the MiniElute column of an RNeasy Mini Kit (Qiagen GmbH). The rest of the procedure was according to the manufactures instruction.

Extracted RNA was reverse transcribed using TATAA GrandScript cDNA Synthesis Kit (Tataa Biocenter) by adding 2 μg total RNA in a final reaction volume of 20 μl in a Biorad CFX96 instrument according to the manufacturers instruction.

The qPCR was performed using TATAA SYBR Grandmaster Mix (Tataa Biocenter) in a Biorad CFX384 instrument with a temperature protocol of 95° C. for 60s, followed by 45 cycles of 95° C. for 5s, 60° C. for 30s and 72° C. for 10s, followed by a melt curve from 55° C. to 95° C. in 0.5° C. steps. For each reaction, 4 μl cDNA was used in a total reaction volume of 20 μl. For the qPCR two assays were done, one directed against human cytochrome B (hCytB) and the other against mouse cytochrome B (mCytB). The qPCR were performed in duplicates for the hCytB assay and in single measurements for the mCytB assay.

### Statistics

All values represented were the average of that experiment and the graphs plotted were the mean of that group and the error bars were the standard error mean of original value. The graphs were plotted using Graph Pad Prism software Version 6.0. Student t-test was used to calculate significant differences between groups. A P value<0.05 was considered significant.

### Example 2: Effect of the Pig Skin Gel (PSG) and Human Peripheral Blood Mononuclear Cells (hPBMC) on Full-Thickness Wound Healing

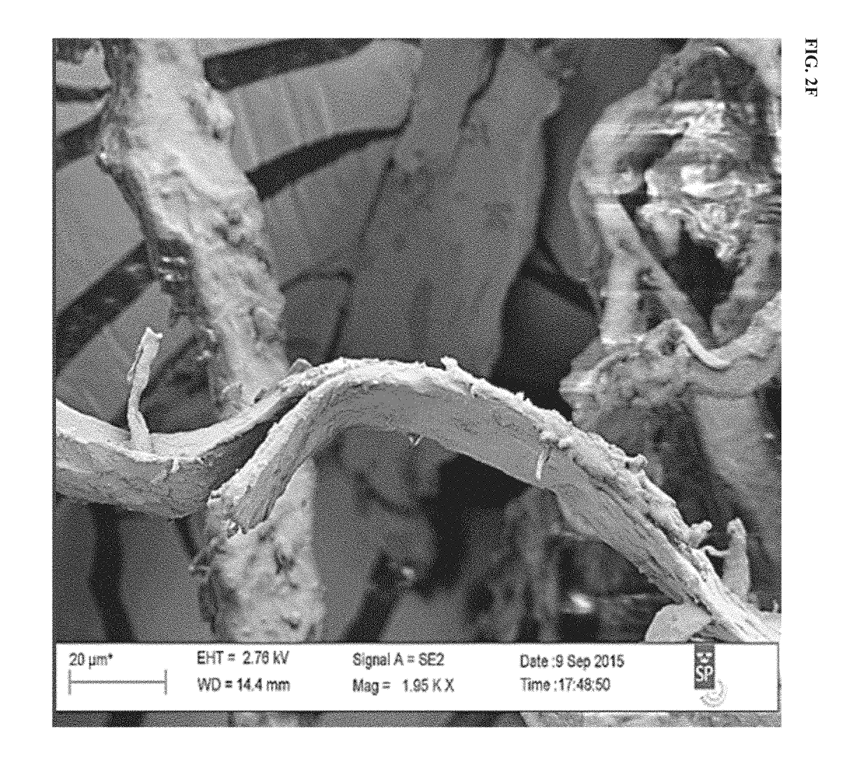

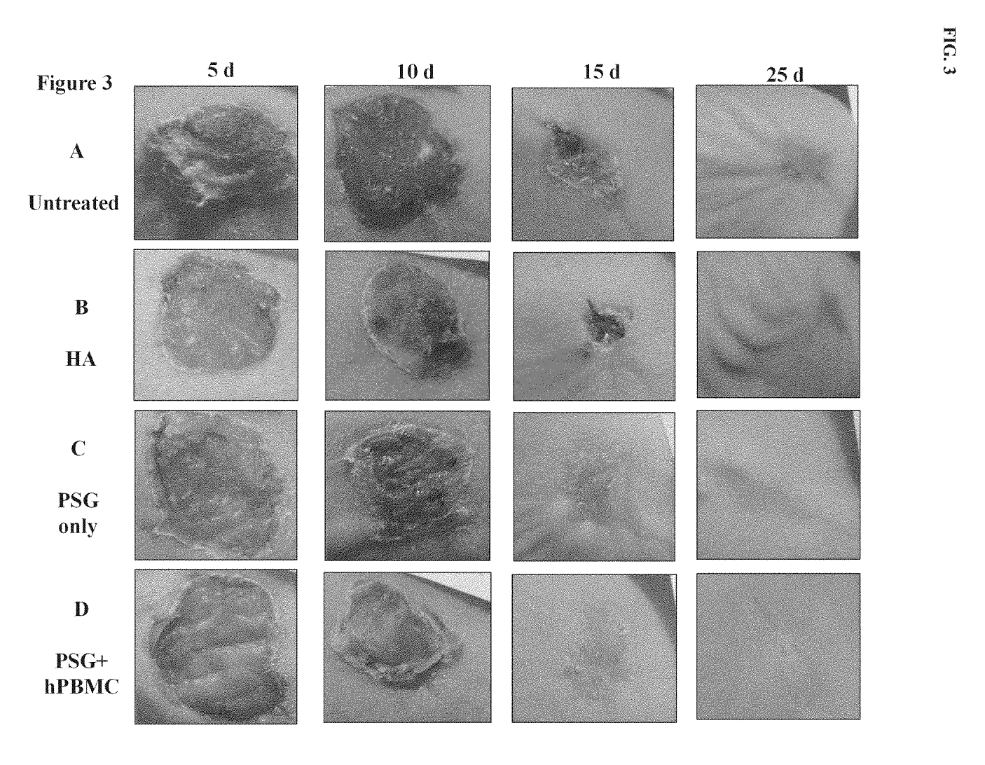

To determine the wound healing capacity of the PSG, acute full-thickness excision wounds created on the back of nude mice were either untreated or treated with HA, PSG only or PSG+hPBMC. FIG. 3 shows the wound healing process in untreated wounds and in wounds after treatment with HA, PSG only and PSG+hPBMC. At 5 and 10 days post-operation, scabs were observed in all groups. However, by day 15, the scabs were peeling off in PSG-treated groups and the wounds were replete with regenerated skin.

Wound Closure in PSG only and PSG+hPBMC Treated Animals

Clear differences were observed with regards to wound closure in animals treated with PSG only and PSG+hPBMC. By 15 days, the wounds in these animals represented the most significant difference in wound closure. Already on day 15, complete wound closure was observed in 5/6 (83%, p<0.05) mice treated with PSG+hPBMC, and in 4/6 (66%, p<0.05) mice treated with PSG only as compared to 0/3 (0%) in untreated and mice treated with HA. On day 25, complete wound healing was observed in all groups. The healed skin in animals treated with PSG only and PSG+hPBMC was similar to normal skin, while an elongated scar was still observed in the healed skin of the control groups due to excessive contraction. As shown in FIG. 3, the difference between the control groups and the PSG-treated was remarkable at 15 days after the operation (p<0.05), but was not significant at 25 days.

### Measurement of Wound Sizes

The wound images were quantified to show the healing areas at different time points. Measurements of wound sizes on day 15, showed that the animals treated with PSG (7.95±1.96 mm²; p<0.05) and PSG+hPBMC (3.5±1.92 mm²; p<0.05) had significantly smaller wound areas as compared to untreated animals (wound area 26.58±4.42 mm²). The wound areas in animals treated with PSG and PSG+hPBMC were also lower when compared to animals treated with HA (13±8.88 mm²) (Table 1).

TABLE 1
Measurement of wound sizes at various time points in untreated and
animals treated with hyaluronic acid (HA), pig skin gel (PSG) or pig
skin gel and human peripheral blood cells (PSG + hPBMC).
Wound size in mm²

| | 0 day | | | | |
|---|---|---|---|---|---|
| | (d) | 5 d | 10 d | 15 d | 25 d |
| Untreated | 122 ± 6.3 | 51 ± 2 | 52 ± 4 | 26 ± 6 | 3 ± 3 |
| HA | | 83 ± 21 | 29 ± 1 | 13 ± 9 | 0 |
| | | | | (p = ns) | |
| PSG only | | 107 ± 7 | 53 ± 5 | 8 ± 2 | 0 |
| | | | | (p = 0.03) | |
| PSG + hPBMC | | 90 ± 5 | 77 ± 8 | 3.5 ± 2* | 0 |
| | | | | (p = 0.02) | |

*As compared to untreated animals

### Wound Healing Process and Structure of Restored Tissue (e.g., Collagen Deposition)

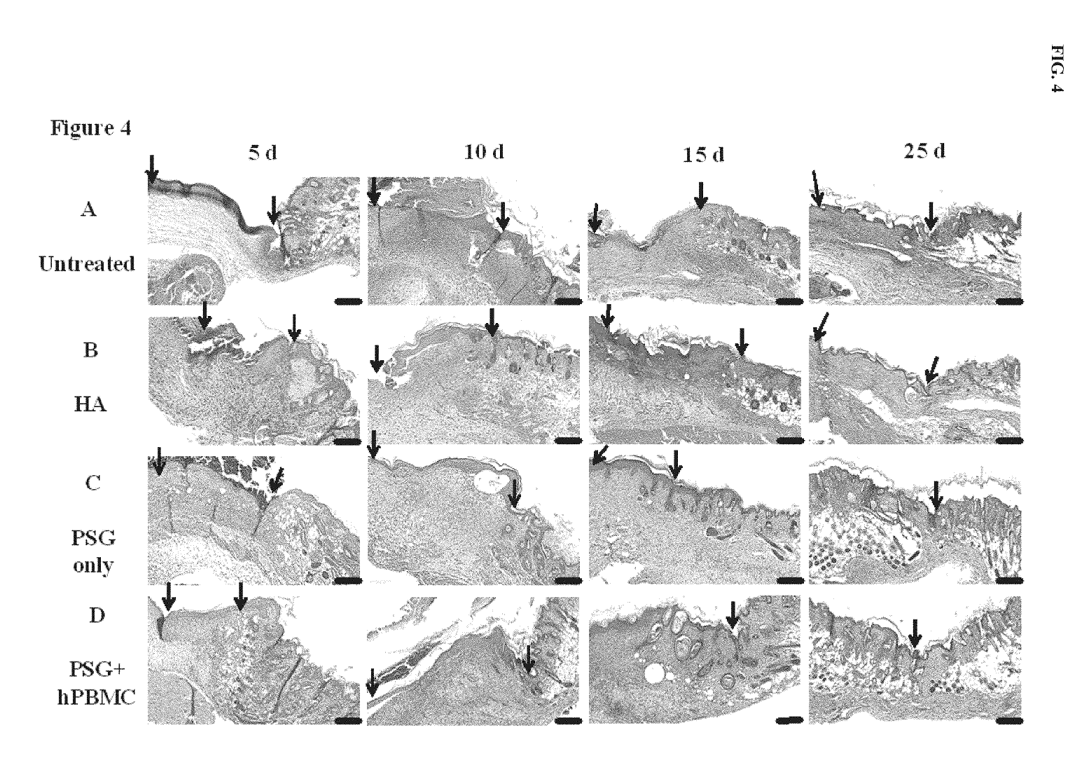

Histological staining was performed to assess the wound-healing processes and the structure of the restored tissues (FIG. 4). Five days after the operation, all the groups exhibited abundant inflammatory cells. Ten days later, wounds in the control groups were discernable from adjacent tissues, and no distinguishable keratin layer was observed. In the PSG and PSG+hPBMC-treated groups, new epidermal cells had migrated around the wound edge and a keratin layer was distinctly noticeable. On days 15 and 25, H&E staining of cross-sections of the healed wounds revealed that the newly formed epidermal layer displayed great similarity to surrounding epidermis with regards to organization and morphology in PSG and PSG+hPBMC-treated wounds as compared to the untreated and HA-treated wounds. Additionally, in the untreated and HA-treated groups, the migration rate was limited and could not bridge the entire wound surface by 25 days, showing a non-keratinized center (FIG. 4).

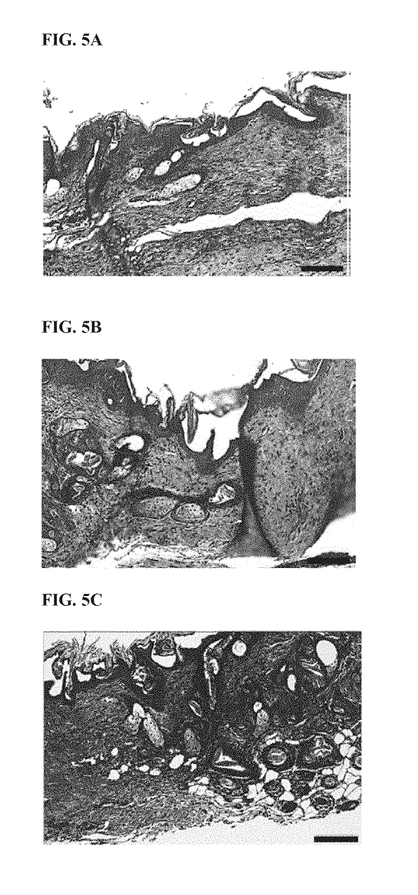

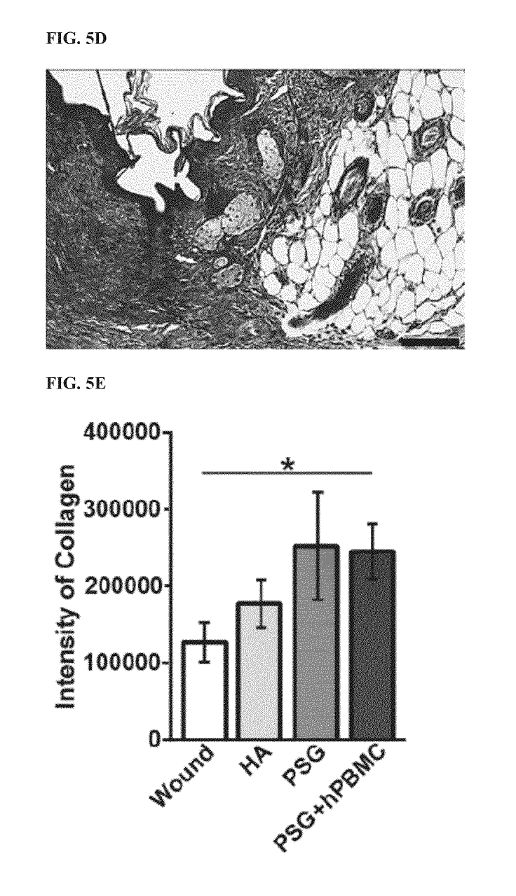

The degree of collagen deposition within the wound bed was assessed using Masson's trichrome staining, as increased, organized collagen deposition is associated with improved wound bed maturation. MT staining showed a markedly increased expression of collagen in the skin biopsies of PSG and PSG+hPBMC-treated animals as compared to controls (FIGS. 5A-5D). In order to confirm the histopathological analysis, quantification of collagen by densitometry was performed in the newly regenerated skin (day 25 after operation). Animals in groups treated with PSG+hPBMC (245011±35832 a.u, p<0.05) had significantly higher amount of collagen as compared to the untreated (127001±25429 a.u) group. Even though collagen density in mice with PSG treatment (252314±70005 a.u, p=0.07) also a showed high value as compared to untreated however, it was not statistically significant. The HA-treated group had 177239±31097 a.u density and was also not significant if compared to untreated group (FIG. 5E).

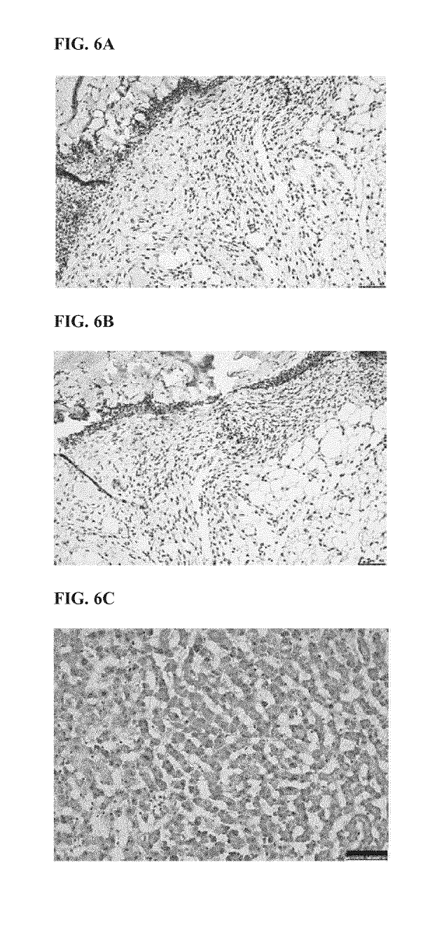

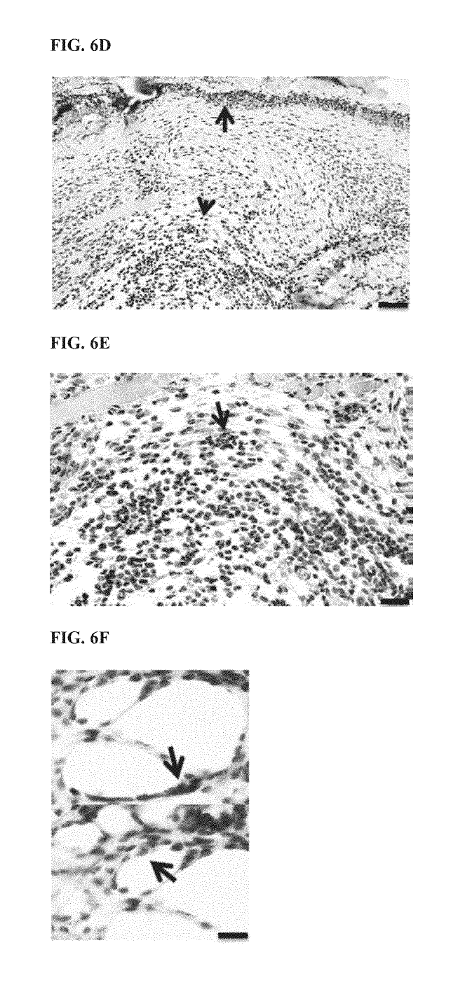

To determine whether human cells in PSG survived after its application on wounds, paraffin sections of partially healed wounds at days 5 and 10 were immunostained with human specific anti-mitochondrial antibodies. The results demonstrated the absence of positively stained cells in the controls (FIGS. 6A and 6B), but the presence of human mitochondrial positive cells in human liver tissue (positive control; FIG. 6C) as well as in both dermal and epidermal layers of animals treated with PSG+hPBMC (FIGS. 6D-6F). These results indicated that human cells accelerated the re-epithelialization of wounded skin by facilitating neovascularization.

### Example 3: Human and Host Blood Vessel Formation within the Wounds Accelerates Wound Healing by Facilitating Neovascularization

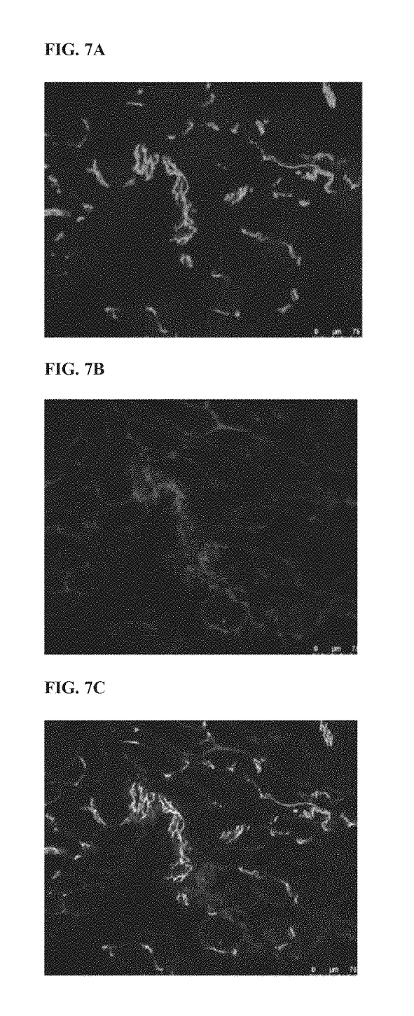

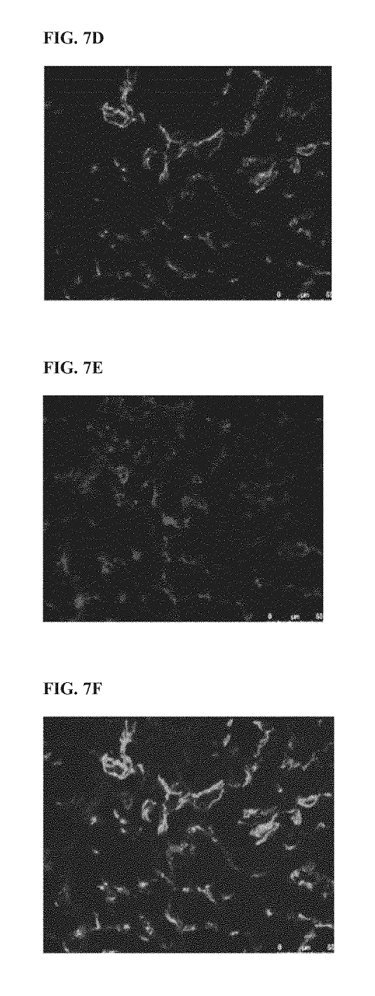

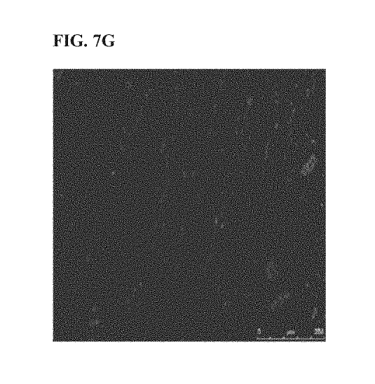

CD31 staining showed vascularization in each experimental group at 5 and 10 days after surgery. A large number of host (mouse) microbloodvessels were observed in the PSG+hPBMC-treated group on day 5 & 10, and the microbloodvessel density was much higher compared with the control group (FIGS. 7A, 7D & 7G respectively). Importantly, when stained with human-specific anti-CD31 antibodies, several small blood vessels stained positive for this marker in the PSG+hPBMC-treated group at day 5 (FIG. 7B). However the number of blood vessels staining positive for human CD31 decreased on day 10 (FIG. 7E) and were negligible on day 15. This finding indicated that the human PBMCs accelerated wound healing by facilitating neovascularization.

### Example 4: Detection of Human RNA in Newly Formed Skin

A total of 38 skin samples taken at days 5, 10, 15 and 25 were analyzed with qPCR for detection of human cells. 23 of the samples were taken from mice treated with hPBMC and 15 samples were from mice not treated with human cells. The Cq values for the mouse qPCR assay were low in all cases ranging between 10 and 14 while the Cq values for the human assay were 20 cycles higher, indicating, as expected, a low number of human cells present in the mouse tissue. Human RNA could be identified in 20 out of the 23 samples treated with human cells while no human RNA was identified in three of the samples. For the samples not treated with human cells, a positive qPCR results with Cq value in similar range was obtained in four of the 15 samples.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

Numbered clauses of the present disclosure are:

1. A composition comprising extracellular matrix (ECM) components of a mammalian tissue and a polymer.

2. The composition of claim 1, wherein the composition is an engineered biomaterial.

3. The biomaterial of claim 2, wherein the biomaterial is a gel composition.

4. The composition of one of claims 1-3, further comprising a growth factor selected from the group consisting of: granulocyte macrophase-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C—C motif) ligand 5), IL-8, IGFs, fibroblast growth factor (FGF)-1, FGF-2, FGF-3, FGF-4, FGF-5, FGF-6, FGF-7, FGF-8, FGF-9, transforming growth factor (TGF)-13, VEGF, platelet-derived growth factor (PDGF)-A, PDGF-B, HB-EGF, hepatocyte growth factor (HGF), tumor necrosis factor (TNF)-α, insulin-like growth factor (IGF)-I, and any combination(s) thereof.

5. The composition of one of claims 1-4, wherein mammalian tissue comprises epithelial and/or connective tissue.

6. The composition of one of claims 1-5, where the mammalian tissue is obtained from skin, placenta, and/or umbilical cord.

7. The composition of one of claims 1-6, wherein the polymer is a carbohydrate based polymer.

8. The composition of claim 7, wherein the polymer is a disaccharide polymer.

9. The composition of claim 8, wherein the disaccharide polymer is hyaluronic acid.

10. The composition of one of claims 1-9, further comprising blood cells or platelet rich plasma.

11. The composition of one of claim 10, wherein the blood cells are peripheral blood mononuclear cells (PBMCs).

12. The composition of claim 11, wherein the PBMCs are human cells.

13. The composition of claim 12, wherein the human PBMCs are autologous.

14. The composition of one of claims 1-13, wherein the ECM components comprise collagen, elastin, and/or sulfated glycosaminoglycans (GAGs).

15. The composition of one of claims 2-14, wherein the biomaterial comprises ribbon-like fibers.

16. The composition of claim 15, wherein the fibers are of about 1 μm-about 40 μm in width, about 0.1 μm-about 10 μm in thickness, and/or about ≥70 μm-≤4000 μm in length.

17. The composition of claim 16, wherein the fibers are of about 5 μm-about 30 μm in width.

18. The composition of claim 17, wherein the fibers are of about 0.5 μm-about 5 μm in thickness.

19. The composition of claim 16, wherein the fibers are of about ≥75 μm-about ≤800 μm in length.

20. The composition of one of claims 1-19, wherein the mammalian tissue is obtained from a mammal selected from the group consisting of: pig, cow, lamb, goat, sheep, and human.

21. The composition of claim 20, wherein the pig is a knockout mutant for α-Gal (Galα1,3-Galβ1-4GlcNAc-R) epitope.

22. A method of healing a wound of a subject in need thereof, comprising applying a composition comprising extracellular matrix (ECM) components of a mammalian tissue and a polymer to a wound of said subject.

23. A method of delivering a therapeutic agent to a wound of a subject in need thereof, comprising applying a composition comprising extracellular matrix (ECM) components of a mammalian tissue, a polymer, and a therapeutic agent to a wound of said subject.

24. A method of delivering a growth factor to a wound of a subject in need thereof, comprising applying a composition comprising extracellular matrix (ECM) components of a mammalian tissue, a polymer, and a growth factor to a wound of said subject.

25. A method of hydrating a wound of a subject in need thereof, comprising applying a composition comprising extracellular matrix (ECM) components of a mammalian tissue and a polymer to a wound of said subject.

26. The method of one of claims 22-25, wherein the composition is an engineered bio material.

27. The method of one of claims 22-25, wherein the biomaterial is a gel composition.

28. The method of claim 24, wherein the growth factor is selected from the group consisting of granulocyte macrophase-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C—C motif) ligand 5), IL-8, IGFs, fibroblast growth factor (FGF)-1, FGF-2, FGF-3, FGF-4, FGF-5, FGF-6, FGF-7, FGF-8, FGF-9, transforming growth factor (TGF)-13, VEGF, platelet-derived growth factor (PDGF)-A, PDGF-B, HB-EGF, hepatocyte growth factor (HGF), tumor necrosis factor (TNF)-α, insulin-like growth factor (IGF)-I, and any combination(s) thereof.

29. The method of one of claims 22-28, wherein the wound is an acute wound or a chronic wound.

30. The method of one of claims 22-28, wherein the wound is a skin wound.

31. The method of one of claims 22-28, wherein the wound is a skin tear, friction, closed impact surgical wound, skin abrasion, burn, skin incision, skin laceration, skin contusion, skin puncture, pressure ulcer, venous ulcers, arterial ulcers, neuropathic/diabetic wounds, lymphedema, or surgical site incision.

32. The method of one of claims 22-31, wherein mammalian tissue comprises epithelial and/or connective tissue.

33. The method of one of claims 22-32, where the mammalian tissue is obtained from skin, placenta, and/or umbilical cord.

34. The method of one of claims 22-33, wherein the polymer is a carbohydrate based polymer.

35. The method of one of claims 22-33, wherein the polymer is a disaccharide polymer.

36. The method of one of claim 35, wherein the disaccharide polymer is hyaluronic acid.

37. The method of one of claims 22-36, wherein the composition further comprises blood cells or platelet rich plasma.

38. The method of claim 37, wherein the blood cells are peripheral blood mononuclear cells (PBMCs).

39. The method of claim 38, wherein the PBMCs are human cells.

40. The method of claim 39, wherein the human PBMCs are autologous.

41. The method of one of claims 22-40, wherein the ECM components comprise collagen, elastin, and/or sulfated glycosaminoglycans (GAGs).

42. The method of one of claims 22-40, wherein the composition comprises ribbon-like fibers.

43. The method of claim 42, wherein the fibers are of about 1 μm-about 40 μm in width, about 0.1 μm-about 10 μm in thickness, and/or about ≥70 μm-≤4000 μm in length.

44. The method of claim 43, wherein the fibers are of about 5 μm-about 30 μm in width.

45. The method of claim 43, wherein the fibers are of about 0.5 μm-about 5 μm in thickness.

46. The method of claim 43, wherein the fibers are of about ≥75 μm-about ≤800 μm in length.

47. The method of one of claims 22-46, wherein the mammalian tissue is obtained from a mammal selected from the group consisting of pig, cow, lamb, goat, sheep, and human.

48. The method of claim 47, wherein the pig is a knockout mutant for α-Gal (Galα1,3-Galβ1-4GlcNAc-R) epitope.

49. The method of one of claims 22-48, wherein the method reduces scar tissue formation.

50. The method of claim 49, wherein reduced scar tissue formation is compared to a wound treated with hyaluronic acid (HA).

51. The method of one of claims 22-50, wherein the method reduces decoloration of the healing wound.

52. The method of claim 51, wherein reduced decoloration is compared to a wound treated with hyaluronic acid (HA).

53. The method of claim 39, wherein the method promotes migration of keratinocytes and epithelial cells.

54. The method of claim 39, wherein the method promotes neovascularization at the wound.

55. The method of claim 54, wherein the neovascularization is promoted in 5 to 10 days after applying the bio material to the wound.

56. The method of claim 54, wherein the neovascularization results in ECM remodeling at the wound.

57. The method of claim 39, wherein reduced elongated scar is observed at the site of the healed wound, compared to a wound treated with hyaluronic acid.

58. The method of one of claims 22-57, wherein the biomaterial generates epidermal cells and restores bilayer structure of the epidermis and dermis.

59. The method of claim 58, wherein the wound is healed within 15-40 days of applying the biomaterial to the wound.

60. The method of claim 59, wherein the wound is healed within 25 days of applying the biomaterial to the wound.

61. A method of preparing a composition comprising extracellular matrix (ECM) components of a mammalian tissue and a polymer, the method comprising decellularzing the mammalian tissue to prepare a powder comprising extracellular matrix (ECM) components, mixing the powder with a medium comprising buffer, nutrients, growth factor, or polymer, wherein the powder soaks up the medium, thereby preparing the composition.

62. The method of claim 61, wherein the powder is prepared by treating the tissue with a chemical, freeze-drying the chemical treated tissue, and homogenization of freeze-dried tissue.

63. The method of one of claims 61-62, wherein the powder is filamentous.

64. The method of one of claims 61-63, wherein the medium is serum containing, reduced serum, or non-serum containing medium.

65. The method of one of claims 61-64, wherein the growth factor is selected from the group consisting of Recombinant 4-1BBL, Recombinant 6Ckine, 6Ckine Recombinant Human Protein, ANGPT2 (ANG2), ANGPTL5, Activin A, Activin Rib, BAFF, BAMBI, CXCL13, BDNF, BLC, BMP2, BMP4, BMP5, BMP7, BMPR1A, CCL1, CCL17, CCL20 (MIP-3), CCL21, CD40, GM-CSF, IL-3, IL-4, NT-6, HB-GAM, MK, IP-10, PF-4, MCP-1, RANTES, IL-8, IGFs, FGF 1, FGF-2, FGF-3, FGF-4, FGF-5, FGF-6, FGF-7, FGF-8, FGF-9, TGF-β, VEGF. PDGF-A, PDGF-B, HB-EGF, HGF, TNF-α, IGF-I, and any combination(s) thereof.

66. The composition of one of claims 1-21, wherein the ECM component is a recombinant ECM component.

67. The composition of claim 66, wherein the recombinant ECM component is an isolated and/or a purified ECM component.

68. The method of one of claims 22-65, wherein the ECM component is a recombinant ECM component.

69. The method of claim 68, wherein the recombinant ECM component is an isolated and/or a purified ECM component.

70. The composition of one of claims 1-21 and 66-67, or method of one of claims 22-65 and 68-69, wherein the mammalian tissue is a decellularized mammalian tissue.

What is claimed is:

1. A composition comprising extracellular matrix (ECM) component of a mammalian tissue and a polymer.

2. The composition of claim 1, wherein the composition is an engineered biomaterial.

3. The composition of claim 2, wherein the engineered biomaterial is a gel composition.

4. The composition of one of claims 1-3, further comprising a growth factor selected from the group consisting of granulocyte macrophase-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C—C motif) ligand 5), IL-8, IGFs, fibroblast growth factor (FGF)-1, FGF-2, FGF-3, FGF-4, FGF-5, FGF-6, FGF-7, FGF-8, FGF-9, transforming growth factor (TGF)-β, VEGF, platelet-derived growth factor (PDGF)-A, PDGF-B, HB-EGF, hepatocyte growth factor (HGF), tumor necrosis factor (TNF)-α, insulin-like growth factor (IGF)-I, and any combination(s) thereof.

5. The composition of one of claims 1-4, wherein mammalian tissue comprises epithelial and/or connective tissue.

6. The composition of one of claims 1-5, where the mammalian tissue is obtained from skin, placenta, and/or umbilical cord.

7. The composition of one of claims 1-6, wherein the polymer is a carbohydrate based polymer.

8. The composition of claim 7, wherein the polymer is a disaccharide polymer.

9. The composition of claim 8, wherein the disaccharide polymer is hyaluronic acid.

10. The composition of one of claims 1-9, further comprising blood cells or platelet rich plasma.

11. The composition of claim 10, wherein the blood cells are peripheral blood mononuclear cells (PBMCs).

12. The composition of claim 11, wherein the PBMCs are human cells.

13. The composition of claim 12, wherein the human PBMCs are autologous.

14. The composition of one of claims 1-13, wherein the ECM components comprise collagen, elastin, and/or sulfated glycosaminoglycans (GAGs).

15. The composition of one of claims 2-14, wherein the biomaterial comprises ribbon-like fibers.

16. The composition of claim 15, wherein the fibers are of about 1 μm-about 40 μm in width, about 0.1 μm-about 10 μm in thickness, and/or about ≥70 μm-≤4000 μm in length.

17. The composition of claim 16, wherein the fibers are of about 5 μm-about 30 μm in width.

18. The composition of claim 16, wherein the fibers are of about 0.5 μm-about 5 μm in thickness.

19. The composition of claim 16, wherein the fibers are of about ≥75 μm-about ≤800 μm in length.

20. The composition of one of claims 1-19, wherein the mammalian tissue is obtained from a mammal selected from the group consisting of: pig, cow, lamb, goat, sheep, and human.

21. The composition of claim 20, wherein the pig is a knockout mutant for α-Gal (Galα1,3-Galβ1-4GlcNAc-R) epitope.

22. A composition according to one of claims 1-21 for use in therapy.

23. A composition comprising extracellular matrix (ECM) component of a mammalian tissue, a polymer, and a therapeutic agent, for use in delivering the therapeutic agent to a wound of a subject in need thereof.

24. A composition comprising extracellular matrix (ECM) component of a mammalian tissue, a polymer, and a growth factor, for use in delivering the growth factor to a wound of a subject in need thereof.

25. A composition comprising extracellular matrix (ECM) component of a mammalian tissue and a polymer, for use in hydrating a wound of a subject in need thereof.

26. The composition for use of one of claims 23-25, wherein the composition is an engineered bio material.

27. The composition for use of one of claims 23-25, wherein the biomaterial is a gel composition.

28. The composition for use of claim 24, wherein the growth factor is selected from the group consisting of: granulocyte macrophase-colony stimulating factor (GM-CSF), interleukin (IL)-3, IL-4, neutrophin (NT)-6, pleiotrophin (HB-GAM), midkine (MK), interferon inducible protein-10 (IP-10), platelet factor (PF)-4, monocyte chemotactic protein-1 (MCP-1), RANTES (CCL-5, chemokine (C—C motif) ligand 5), IL-8, IGFs, fibroblast growth factor (FGF)-1, FGF-2, FGF-3, FGF-4, FGF-5, FGF-6, FGF-7, FGF-8, FGF-9, transforming growth factor (TGF)-β, VEGF, platelet-derived growth factor (PDGF)-A, PDGF-B, HB-EGF, hepatocyte growth factor (HGF), tumor necrosis factor (TNF)-α, insulin-like growth factor (IGF)-I, and any combination(s) thereof.

29. The composition for use of one of claims 22-28, wherein the wound is an acute wound or a chronic wound.

30. The composition for use of one of claims 22-28, wherein the wound is a skin wound.

31. The composition for use of one of claims 22-28, wherein the wound is a skin tear, friction, closed impact surgical wound, skin abrasion, burn, skin incision, skin laceration, skin contusion, skin puncture, pressure ulcer, venous ulcers, arterial ulcers, neuropathic/diabetic wounds, lymphedema, or surgical site incision.

32. The composition for use of one of claims 23-31, wherein mammalian tissue comprises epithelial and/or connective tissue.

33. The composition for use of one of claims 23-32, where the mammalian tissue is obtained from skin, placenta, and/or umbilical cord.

34. The composition for use of one of claims 23-33, wherein the polymer is a carbohydrate based polymer.

35. The composition for use of one of claims 23-33, wherein the polymer is a disaccharide polymer.

36. The composition for use of claim 35, wherein the disaccharide polymer is hyaluronic acid.

37. The composition for use of one of claims 22-36, wherein the composition further comprises blood cells or platelet rich plasma.

38. The composition for use of claim 37, wherein the blood cells are peripheral blood mononuclear cells (PBMCs).

39. The composition for use of claim 38, wherein the PBMCs are human cells.

40. The composition for use of claim 39, wherein the human PBMCs are autologous.

41. The composition for use of one of claims 23-40, wherein the ECM components comprise collagen, elastin, and/or sulfated glycosaminoglycans (GAGs).

42. The composition for use of one of claims 23-40, wherein the composition comprises ribbon-like fibers.

43. The composition for use of claim 42, wherein the fibers are of about 1 μm-about 40 μm in width, about 0.1 μm-about 10 μm in thickness, and/or about ≥70 μm-≤4000 μm in length.

44. The composition for use of claim 43, wherein the fibers are of about 5 μm-about 30 μm in width.

45. The composition for use of claim 43, wherein the fibers are of about 0.5 μm-about 5 μm in thickness.

46. The composition for use of claim 43, wherein the fibers are of about ≥75 μm-about ≤800 μm in length.

47. The composition for use of one of claims 23-46, wherein the mammalian tissue is obtained from a mammal selected from the group consisting of: pig, cow, lamb, goat, sheep, and human.

48. The composition for use of claim 47, wherein the pig is a knockout mutant for α-Gal (Galα1,3-Galβ1-4GlcNAc-R) epitope.

49. The composition for use of one of claims 22-48, wherein the method reduces scar tissue formation.

50. The composition for use of claim 49, wherein reduced scar tissue formation is compared to a wound treated with hyaluronic acid (HA).

51. The composition for use of one of claims 22-50, wherein the method reduces decoloration of the healing wound.

52. The composition for use of claim 51, wherein reduced decoloration is compared to a wound treated with hyaluronic acid (HA).

53. The composition for use of claim 39, wherein the method promotes migration of keratinocytes and epithelial cells.

54. The composition for use of claim 39, wherein the method promotes vascularization or neovascularization at the wound.

55. The composition for use of claim 54, wherein the vascularization or neovascularization is promoted in 5 to 10 days after applying the biomaterial to the wound.

56. The composition for use of claim 54, wherein the vascularization or neovascularization results in ECM remodeling at the wound.

57. The composition for use of claim 39, wherein reduced elongated scar is observed at the site of the healed wound, compared to a wound treated with hyaluronic acid.

58. The composition for use of one of claims 22-57, wherein the biomaterial generates epidermal cells and restores bilayer structure of the epidermis and dermis.

59. The composition for use of claim 58, wherein the wound is healed within 15-40 days of applying the biomaterial to the wound.

60. The composition for use of claim 59, wherein the wound is healed within 25 days of applying the biomaterial to the wound.

61. A method of preparing a composition comprising extracellular matrix (ECM) component of a mammalian tissue and a polymer, the method comprising decellularzing the mammalian tissue to prepare a powder comprising extracellular matrix (ECM) components, mixing the powder with a medium comprising buffer, nutrients, growth factor, or polymer, wherein the powder soaks up the medium, thereby preparing the composition.

62. The method of claim 61, wherein the powder is prepared by treating the tissue with a chemical, freeze-drying the chemical treated tissue, and homogenization of freeze-dried tissue.

63. The method of one of claims 61-62, wherein the powder is filamentous.

64. The method of one of claims 61-63, wherein the medium is serum containing, reduced serum, or non-serum containing medium.

65. The method of one of claims 61-64, wherein the growth factor is selected from the group consisting of Recombinant 4-1BBL, Recombinant 6Ckine, 6Ckine Recombinant Human Protein, ANGPT2 (ANG2), ANGPTL5, Activin A, Activin Rib, BAFF, BAMBI, CXCL13, BDNF, BLC, BMP2, BMP4, BMP5, BMP7, BMPR1A, CCL1, CCL17, CCL20 (MIP-3), CCL21, CD40, GM-CSF, IL-3, IL-4, NT-6, HB-GAM, MK, IP-10, PF-4, MCP-1, RANTES, IL-8, IGFs, FGF 1, FGF-2, FGF-3, FGF-4, FGF-5, FGF-6, FGF-7, FGF-8, FGF-9, TGF-β, VEGF. PDGF-A, PDGF-B, HB-EGF, HGF, TNF-α, IGF-I, and any combination(s) thereof.

66. The composition of one of claims 1-22, wherein the ECM component is a recombinant ECM component.

67. The composition of claim 66, wherein the recombinant ECM component is an isolated and/or a purified ECM component.

68. The composition for use of one of claims 23-60, wherein the ECM component is a recombinant ECM component.

69. The composition for use of claim 68, wherein the recombinant ECM component is an isolated and/or a purified ECM component.

70. The composition of one of claims 1-60 and 66-69, or method of one of claims 61-65, wherein the mammalian tissue is a decellularized mammalian tissue.

71. The composition according to claim 22, wherein the composition is for use in healing a wound in a subject in need thereof.
